(19) United States
(12) Patent Application Publication
(10) Pub. No.: US 2019/0201586 A1
(43) Pub. Date: Jul. 4, 2019

(54) Three-Dimensional Tissue Matrix Scaffold System

(71) Applicant: Washington State University, Pullman, WA (US)

(72) Inventors: Weimin Li, Spokane Valley, WA (US); Girdhari Rijal, Spokane, WA (US)

(21) Appl. No.: 16/312,062

(22) PCT Filed: Jun. 23, 2017
(86) PCT No.: PCT/US17/39135

§ 371 Date: Dec. 20, 2018

Related U.S. Application Data
(60) Provisional application No. 62/354,335, filed on Jun. 24, 2016.

(51) Int. Cl. A61L 27/56; A61K 38/18; A61L 27/12; C12N 5/00; C12Q 1/02; C12N 5/071; C12N 5/077

(52) U.S. Cl. CPC A61L 27/56; A61K 38/18; A61L 27/12; C12N 2533/90; C12Q 1/025; C12N 5/0629; C12N 5/0656; C12N 5/0062

(57) ABSTRACT

Provided are porous, hydrogel, and multilayer tissue matrix scaffolds that are derived from native tissues. The scaffolds can be used for cell culture, preparing tumoroids for in vivo implant, and testing or screening the efficacies or toxicities of drugs toward cancers or other diseases.

### CROSS-REFERENCE TO RELATED APPLICATION

This application claims the benefit of U.S. Provisional Patent Application No. 62/354,335 filed Jun. 24, 2016 which is incorporated herein by reference in its entirety as if fully set forth herein.

### FIELD OF THE DISCLOSURE

The present embodiments herein relate to the field of tissue culture using extracellular matrix (ECM) protein(s), and more particularly to a novel system designed to fabricate tissue matrix scaffold (TMS) derived from native animal tissues while preserving most of the ECM proteins. The innovative design, fabrication process, applications in research and drug/biomarker screening, and regenerative medicine are presented.

### BACKGROUND

Human cells live in a three-dimensional (3D) tissue environment that has multiplex cell populations in addition to a hypoxic condition (lung tissue has the highest oxygen level at about 14%). This tissue microenvironment is essential for cell survival and biological functions (e.g. inter- and intracellular signaling transduction/molecular interactions) in response to extracellular stimuli. It is technically challenging to model human tissue in culture and implant the culture back into live body for disease studies and tissue regeneration.

In particular, the canonical planar tissue culture models and the currently available scaffold culture systems using synthetic polymers or a single component of the extracellular matrix (ECM) do not resemble the support for cell growth in tissue microenvironments. Therapeutic studies and applications based on these methods have yielded inconsistent results and have not demonstrated convincing efficacies in biomarker or drug testing and screening.

In addition, synthetic polymer scaffolds could lead to post-degradation toxicity (e.g. acidic products) to the cells. This not only affects cellular biological functions, but also impairs natural cell-cell and cell-ECM interactions that result in defects in secretion of biomolecules/enzymes critical for cell growth, rejection of the scaffold graft, to cell death or other severe side effects.

Hence, there is an urgent need for the “next generation” of biocompatible and biodegradable scaffolds that can satisfy the needs for scientific research, preclinical and clinical applications.

### SUMMARY

The present disclosure describes the fabrication of porous and hydrogel scaffolds from animal or human tissue ECM extract using an integrated freeze-drying and physiochemical cross-linking method as well as applying a natively present enzyme for cross-linking. The present disclosure also describes assemblies of the porous and the hydrogel scaffolds into multilayered compartmental culture platforms. The scaffold systems described herein are collectively termed as Tissue Matrix Scaffold (TMS). As demonstrated herein, the TMS faithfully mimics native tissue environments both in culture and in animals.

The present disclosure describes compositions including delipidated and decellularized ECM extract. The protein content of the ECM includes various types of collagen, less than about 45% laminin, and other native tissue ECM proteins. The composition can be in the form of a powder, a solution, or a gel, which can be reconstituted to form a TMS. TMS can be a hydrogel TMS or a porous TMS.

The present disclosure also describes methods of making and using the TMS. The TMS can be used as a two-dimensional or three-dimensional cell culture environment to culture cells or tissues. In embodiments, the cultured cells can be implanted in an animal. In particular embodiments, the cultured cells can be implanted in a mammal. The TMS can also be used for drug or toxicological screening and for generating tissues, such as breast tissue, skin tissue, and other tissues or organs.

The present disclosure also describes kits for making and using the TMS.

### BRIEF DESCRIPTION OF THE DRAWINGS

Many of the drawings submitted herein are better understood in color, which is not available in patent application publications at the time of filing. Applicant considers the color versions of the drawings as part of the original submission and reserves the right to present color images of the drawings in later proceedings.

FIGS. 1a-1e TMS fabrication and structural property characterization. (1a) The workflow of the porous TMS fabrication. 1) Collection of breast tissues from 8-12 weeks old mice. 2) Decellularization of the native tissues to produce ECM. 3) Lyophilization of the ECM at −50° C. 4) Enzymatic digestion of the ground ECM in acidic pH. 5) Neutralization of the acidic ECM solution generated hydrogel. 6) Loading the hydrogel into the spherical moulds. 7) Formation of the pre-scaffolds in the moulds at −80° C. 8) Lyophilization of the pre-scaffolds. 9) Formation of the porous scaffolds in the moulds. 10) Treatment of the scaffolds with absolute ethanol and cross-linking the ECM proteins under UV light. 11) Lyophilization of the scaffolds to remove the ethanol. 12) Characterization of the finished TMS scaffolds. A microscopic view of the TMS cross sections after H&E staining was exhibited (scale bar=100 μm). (1b) Comparison of the composition of the decellularized tissues with that of the native tissues at DNA and major ECM protein levels. Error bars represent the s.d. of the measurements of 3 independent batches of the ECM samples. (1c) Characterization of the TMS porosity using scanning electron microscopy (SEM). Different amounts of the lyophilized ECM powder were used to generate TMSs with different pore sizes. (1d) Histological comparison of the cross sections of the blank and the cell-laden decellularized breast tissue-TMSs (DBT-TMSs) generated using decellularized native breast tissues from mice. (1e) Comparison of the occupancies of the cells grown inside the DBT-TMS with that of the native cells lived in mouse breast tissues. The left panels exhibit the close-up views of the H&E stained cross sections of the fibroblasts-laden TMS and the mammary fat-pad tissues. The top-right panel displays a SEM image showing the occupancies of the MDA-MB-231 (MM231) cells on the surface and within the porous TMS. The bottom-right panel shows the distribution patterns of the MM231 cells and stromal cells immunostained with Ki-67 and HER2, respectively, on the cross sections of mouse breast tumors originated from the MM231 cell-laden TMSs. DAPI was used to stain the nuclei of the cells. The red and the yellow arrows indicate stromal and the MM231 cells, respectively. FIGS. 1c-e, scale bars=100 μm.

FIG. 2. Comparison of the differences of the major proteins identified from TMS/mouse mammary tissue ECM vs. those from IrECM hydrogel (Matrigel). The ECM proteins listed in Table 1 and Table 2 (on pages 28-30) were grouped and compared side-by-side. The spectrum counts of the different subunits or chains of a same protein were added up to represent the relative abundance of the functional protein within the ECM.

FIGS. 3a-3n. Cell survival and proliferation in TMS. (3a) Macroscopic and microscopic views of the blank and cell-laden porous DBT-TMSs. Scale bars=1 mm for the macroscopic views and the microscopic views of the H&E stained cross sections; 200 μm for the regional blowups of the H&E stained cross sections. (3b) Proliferation of MCF10A and MM231 cells grown on the DBT-TMSs over a period of 14 days. Error bars represent s.d. of the means of the values from 3 independent experiments. *P<0.01; **P<0.001: compared to the 1^{st} day culture. (3c-3f) The proliferation and distribution of the MM231 cells on the DBT-TMSs were examined on the cross sections of the scaffolds using H&E staining coupled with light microscopy. Scale bar=100 μm. (3g-3j) Live/Dead Cell assays showing robust survival and proliferation of the MM231 cells on the DBT-TMSs over time. Scale bar=100 μm. (3k-3n) Comparison of MCF10A and MM231 cell proliferation profiles on different 3D scaffolds within the defined time frame. Error bars represent s.d. of the means of 3 independent experiments. **P<0.01: compared to the proliferation profiles on the PCL/PLGA scaffolds; ^{#}P<0.05: compared to the proliferation profiles on the collagen scaffolds.

FIGS. 4a-4d Cancer cell growth on the porous synthetic polymer-based scaffolds. MM231 cells seeded and cultured on the PLGA scaffolds were characterized for growth and distribution status (4a-4d) using H&E staining of the cross sections of the scaffolds besides the proliferation analysis demonstrated in FIG. 2k-n. Scale bars=100 μm.

FIGS. 5a-5i. Compartmental 3D tissue culture using the TMS system. (5a) Generation of the multilayered/compartmentalized TMS culture system. MM231 cells were cultured on the porous DBT-TMS, followed by either covering with a layer of blank TMS hydrogel or directly placing into culture for in vitro or in vivo experiments. Hydrogel premixed with another type of cells different from those coated on the porous TMS was applied outside the first layer, and enzymatically cross-linked, forming a second gel layer. The multilayered TMS assembly was then subjected to culture or/and implantation into animals for further analysis or applications. (5b) H&E staining of the cross sections of a TMS coated with MM231 cells and a layer of hydrogel. (5c) H&E staining of the cross sections of a multilayered TMS containing the porous TMS core coated with MM231 cells and two hydrogel layers with the second gel layer containing the human GM637 fibroblasts. The middle region outlined by dotted lines was a blank hydrogel layer. (5d) DAPI staining of the cross sections of the compartmentally cultured cells grown in the multilayered TMS after 3 days of culture (as shown in c). (5e) IF microscopic view of Ki-67 and HER2 staining on the cross sections of the compartmental TMS samples. Selected regional blowups of the Ki-67 and HER2 staining were shown as insets. (5f-5i) Live/Dead cell staining of the cross sections of the compartmentally cultured MM231 cells (on the porous TMS, right side to the blank hydrogel layer) and the human GM637 fibroblasts (within the second hydrogel layer, left side to the blank hydrogel layer) at different time points of the cultures. Scale bar=100 μm.

FIGS. 6a-6i. Monitor tumor growth with x-ray-based imaging. Computerized Tomography (CT) images of the tumors originated from the 3D scaffolds coated with or without the MM231 cancer cells in the presence or absence of an outer DBT hydrogel layer were taken 3 weeks post-implantation of the scaffolds into the animals. Scale bars=4 mm.

FIG. 7a-7c. Characterization of TMS support for tumor formation in animals. (7a) Evaluation of the biodegradability of the scaffolds and their supports on the MM231 cell-originated tumor development (dissection microscopy images). Scale Bar=4 mm. (7b) Quantification of the sizes of the tumors formed from the different MM231 cell-laden scaffolds. The plotted values reflect the ex-vivo measurements of the tumors. The error bars represent the s.d. of the sizes of three individual tumors of the same implantation background. *P<0.05, **P<0.01: significance of the comparison between the indicated sample groups. (7c i-iv) H&E staining of the cross sections of the tumors originated from the MM231 cell-laden DBT-TMS and DMM231 scaffolds with or without hydrogel coverage. The tumor ECM structure, cell distribution and capillaries (containing the stained red blood cells) are demonstrated. (7c v-viii) IF staining of Ki-67 (green) and HER2 (red) on the tumor cross sections. The cell nuclei were stained with DAPI (blue). 7c i-viii, scale Bars=100 μm.

FIGS. 8a-8h. Tumor development from the cancer cell-laden scaffolds in mice mammary tissues. (8a-8d) H&E staining of the cross sections of the tumors derived from the DBT-TMS and DMM231 scaffolds that carried the MM231 cells in the presence or absence of a hydrogel layer outside the seeded cells. The ECM architectures, overall cell distribution and microvessels within the tumors are displayed. The blowup views of the stained sections were demonstrated in FIG. 7c i-iv. (8e-8h) IF staining of the tumor sections from the experimental groups demonstrates the distribution of the fast-proliferating cells, mostly the cancer cells and the infiltrated stromal cells that are positive for Ki-67, and the HER2 positive cells, such as the normal fibroblasts and endothelial cells, etc. Cell nuclei were stained with DAPI. The blowup views of the stained sections were demonstrated in FIG. 7c v-viii. 8a-8h, scale bars=100 μm.

FIG. 9. Quantification of the capillary and tumor tissue areas on the cross sections of the tumors. H&E stained cross sections of the tumors were analyzed for the occupancies of the blood vessels and the solid tumor tissues as described in the methods. Error bars represent the s.d. of the means of the values from three consecutive slides (per tumor) of three replicate tumors. **P<0.01: comparisons of the counterpart samples between the different scaffold groups or the samples within the same scaffold group in the presence or absence of the hydrogel coverage.

FIGS. 10a-10c. Histological examination of the tumors developed from the cancer cells grown on the PLGA scaffolds. H&E staining of the cross sections of the implanted PLGA scaffolds with or without the MM231 cells and a hydrogel outer layer showed underdeveloped and disorganized ECM structures as well as limited cell infiltration into the ECM. Scale bars=100 μm.

FIGS. 11a-11b. (11a) The proliferation of T47D and BT474 cells grown on the different scaffolds was evaluated with cell proliferation assays. The analysis of the data collected from the indicated time points served as a reference for the drug tests shown in FIGS. 12a-12c. Error bars represent the s.d. of the means of 3 independent experiments. (11b) Evaluation of the cell proliferation in response to Taxol or HT treatment in 2D cultures. Error bars represent the s.d. of the means of 3 independent experiments. **P<0.01: comparison of the average cell proliferation (Day 8^{th}-14^{th}) between the drug treated groups and the non-treated control groups. [symbol] =post-treatment recovery measurement.

FIGS. 12a-12c. Comparison of the sensitivities of the cancer cells grown on the different scaffolds to anticancer drugs. (12a) The impact of the anticancer drugs on cell growth and proliferation supported by the DBT-TMS, collagen, IrECM, and PLGA scaffolds was analyzed and compared. The drug administration pattern and the cell proliferation measurements were detailed in the methods. The error bars represent the s.d. of three independent experiments. The black and green lines within the plot area indicate the comparison of the average T47D or BT474 cell proliferation (Day 8^{th}-14^{th}) between the drug treated groups and the non-treated control groups. The red lines indicate the comparison of the average cell proliferation between the Collagen or PLGA scaffold groups and the DBT-TMS groups. **P<0.01; [symbol] =post-treatment recovery measurement. (12b) Proliferation/inhibition curve plots. The mean values (from 3 independent experiments) of the cell proliferation status on the different scaffolds at the time points Day 1^{st} (the start date of cell proliferation), 8^{th} (1 day after the first treatment), 14^{th} (1 day after the last treatment) and 21^{st} (the end of recovery) as shown in (12a) were plotted. (12c) The clinical trends of tumor development and response to treatment. The tumor growth over the phase of cell proliferation status in response to therapeutic interventions was depicted. The dotted arrows indicate the additional treatment options during the disease progression.

FIGS. 13a-13j. Cancer cell survival and growth status on TMS after drug treatment. In addition to the proliferation assays shown in FIGS. 5a-5i, the sensitivities of T47D or BT474 cells grown on the DBT-TMSs to HT or Taxol treatment were inspected using Live/Dead Cell staining at different time points. Only the T47D/TMS±HT results were shown here, with the similar patterns observed in the BT474/TMS±HT and the T47D/TMS±Taxol or the BT474/TMS±Taxol samples (data not shown). The green and the red signals indicate the live and the dead cells, respectively. Scale bars=100 μm.

FIGS. 14a-14j. Cancer cell survival and growth status on PLGA scaffolds after drug treatment. The sensitivities of the T47D or BT474 cells grown on the PLGA scaffolds to the anticancer drugs were assessed with Live/Dead Cell staining at different time points. The results of the T47D/PLGA±HT samples were displayed, with the similar patterns observed in the BT474/PLGA±HT and the T47D/PLGA±Taxol or the BT474/PLGA±Taxol samples (data not shown). Scale bars=150 μm.

FIGS. 15a, 15b. Flow chart of the two methods for hydrogel and porous TMS generation using animal tissues. (15a) Method for small scale TMS generation, where low amount of fatty tissues is present in the animal tissues, pepsin digestion of the ECM, and UV-light or tyrosinase cross-linking of the TMS porous or hydrogel scaffolds. (15b) Method for large scale TMS production, where high amount of fatty tissues is present, Triton X-100 and lipase decellularization and dilapidation, ECM protein extraction and dialysis, and temperature-aided polymerization of the protein gel for the production of the porous or hydrogel TMS scaffolds (see details in Example 1: Materials and Methods, where TMS fabrication is described).

FIG. 16. TMS multi-well insert and plate design for tissue culture and drug screening. The frame/pillar well inserts carrying round, square, or other shapes of porous or hydrogel TMS are uniquely designed for tissue culture or drug screening using multi-well culture plates. The well inserts can be individual or panelized.

FIGS. 17a-17j. Immunofluorescence staining of biomarker expression in cells grown on various scaffolds.

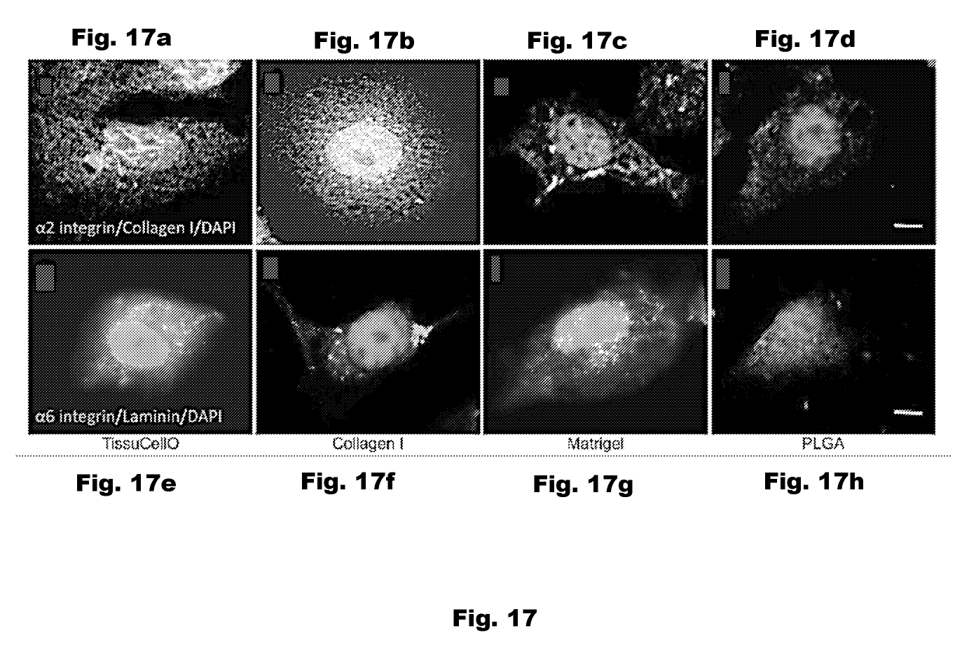

FIG. 18. Techniques for TMS-based bioassays and drug or toxicological screening (top) and multilayer TMS scaffolds or uniform TMS assembly supports to model breast tissue, skin, and other tissues (bottom).

### DETAILED DESCRIPTION

The disclosure describes a method of mimicking the complex tissue microenvironment for tumor growth in culture. In embodiments, the disclosure describes the TMS system fabricated using unique methods for the extraction of native human or animal tissues ECM, the cross-linking of the extracts for the reconstitution of ECM, and the biomedical research and pharmaceutical applications. TMS is generated in both porous and hydrogel formats, which allows the construction of multilayered scaffolds for compartmental culture of different cells. Normal and cancerous cells simultaneously cultured in separate layers of TMS were easily distinguished by immunofluorescence staining of biomarkers, and observed for the phenotypes, such as migration and invasion, in the same system. Cancer cells grown on TMS displayed superior proliferation in 3D cultures and tumor formation in animals, and were the least inhibited by the select anticancer drugs with enhanced post-treatment recovery compared to those cultured on planar substratum or other types of scaffolds tested. The method described herein better represents the data from animal model and clinical studies. Importantly, cells grown on the ECM extracted from the native tissues that are source materials for TMS generation displayed distinguished expression of cell surface receptors compared to those cultured on the collagen, Matrigel, and PLGA substrata. The expression of the cellular receptors and other biomolecules are critical for many biological functions and phenotypes of the cells. The TMS system described herein is a new generation of native ECM-based 2D and 3D culture model with broad versatility for research and therapeutic applications.

As is well-known, there exists various synthetic polymer scaffolds. As the name indicates, the term “synthetic polymer scaffold” refers to a structural 3D scaffold with synthetic polymers that provide structure to the scaffold. Examples of synthetic polymers that can be used in synthetic polymer scaffolds include polyethylene glycol (PEG), polycaprolactone (PCL) and poly(lactic-co-glycolic) acid (PLGA).

Presented herein are tissue matrix scaffolds (TMS or TMSs), and kits and reagents for producing and using TMS. Unless otherwise indicated, TMS can refer to porous TMS, hydrogel TMS, a combination of a porous and a hydrogel TMS, and/or multilayer TMS. The TMS described herein do not include synthetic polymers. The term “TMS” refers to both “tissue matrix scaffold” and “tissue matrix scaffolds.”

Also presented are extracellular matrix (ECM) extracts that can be used to produce TMS.

The TMS or ECM extract is derived from one or more fresh tissue samples. The TMS or ECM extract are prepared or obtained from one or more native tissue samples. A native tissue or naturally-occurring tissue can refer to a tissue that obtained or isolated from one or more animals. In embodiments, the tissue(s) is/are from one or more animals. In particular embodiments, the tissue is from one or more mammals, such as human, mouse, pig, cow and/or non-human primate. In embodiments, the TMS or ECM extract is derived from any tissue type that includes an extracellular matrix. In embodiments, the TMS or ECM extract is derived from mammary, adipose, skin, muscle, heart, liver, lung, stomach, kidney, intestine, spleen, pancreas, brain, prostate, blood vessel, bone, tooth, tendon, ligament, endometrium, womb, mucosa, umbilical cord or umbilicus, embryo, or membranous tissue. In particular embodiments, the TMS or ECM extract is derived from non-cancerous, tumor, or cancer tissue.

In embodiments, the TMS or ECM extract includes components of an extracellular matrix that are derived from a tissue. An ECM is a collection of molecules secreted by cells that provide structural and biochemical support to the surrounding cells. ECMs produced by animals include interstitial matrices and basement membranes, which include distinct compositions of ECM molecules. Interstitial matrices are present between various types of cells and can transmit cell-to-cell signals. Basement membranes are sheet-like deposits of ECM that can support growth of epithelial or other types of cells.

ECMs include a variety of proteins and glycosaminoglycans (GAGs), which are long unbranched polysaccharides with repeating disaccharide units. Types of proteins present in the ECM include glycoproteins and proteoglycans. A glycoprotein is any protein with one or more carbohydrate attachment. A proteoglycan is any protein with one or more glycosaminoglycan attachment. Examples of glycoprotein/proteoglycans present in ECM include collagen, laminin, periostin, fibrinogen, fibronectin, nidogen, perlecan, tenascin, EMILIN, and lumican. Other proteins present in the ECM include elastin, titin, perilipin, dermatopontin, vitronectin, antitrypsin, and fibulin.

In embodiments, the TMS or ECM extract includes GAGs. Examples of GAGs present in the ECM include hyaluronic acid and keratin sulfate. In embodiments, the glycosaminoglycan content of a TMS or an ECM can be measured using a 1, 9 dimethylmethylene blue assay.

In embodiments, the TMS or ECM extract includes collagen. Collagen is a structural component of ECM and is the most abundant protein in animal and human ECM. Currently, there are 44 different human collagen genes, which together can encode at least 28 different forms of collagen fibrils. The different protein types of collagen fibril include type I collagen through type XXVIII collagen. Many types of collagen fibril (e.g., type-I) contain two identical alpha chains (e.g., two alpha-1 chains) and one additional alpha chain (e.g., one alpha-2 chain). Type I collagen is the most abundant protein present in interstitial matrix ECM, whereas type IV collagen is the most abundant protein present in basement membrane ECM. Collagen proteins contain up to hundreds of repeat sequences of Gly-Pro-X or Gly-X-HyP, where HyP refers to hydroxyproline. In embodiments, the collagen content of a tissue, a TMS, or an ECM can be measured by a hydroxyproline assay, or by mass spectrometry.

In embodiments, the TMS or ECM extract is collagen-rich. In embodiments, collagen (including any of types 1-XXVIII) is the most abundant protein in the TMS or ECM extract. In embodiments, the TMS or ECM extract contains a collagen content (as a percentage of the total protein content) of about at least 1%, at least 2%, at least 3%, at least 4%, at least 5%, at least 10%, at least 20%, at least 30%, at least 40%, at least 50%, at least 60%, at least 70%, at least 80%, or at least 90%. In embodiments, the collagen content (as a percentage of the total protein content) is about 50-95%, 60-90%, 70-85%, 70-80%, 70-90%, or 70-95%. In embodiments, the collagen content (as a percentage of the total protein content) is less than about 50%, less than 40%, less than 30%, less than 20%, less than 10%, less than 5%, less than 4%, less than 3%, less than 2%, or less than 1%.

In particular embodiments, type I collagen is the most abundant type of collagen in the TMS or ECM extract. In particular embodiments, the collagen content is at least 30%, at least 40% or at least 50% type I collagen.

In embodiments, the TMS or ECM extract includes laminin. Laminin is a major component of basement membrane ECM, and forms connections with various cell surface receptors, such as integrins. In particular embodiments, the laminin content (as a percentage of the total protein content) in the TMS or ECM extract is less than about 50%, less than about 45%, less than 40%, less than 30% less than 20%, less than 10%, or less than 5%.

In embodiments, the TMS or ECM extract retains the molecular components of the ECM from which the tissue is derived. In embodiments, the TMS includes a collagen content that is about at least 50%, at least 60%, at least 70%, at least 80%, or at least 90% of the collagen content of the tissue from which the extract is derived. In particular embodiments, the TMS includes a GAG content of about at least 40%, at least 50% or at least 60% of the GAG content of the tissue from which the extract is derived.

In embodiments, the TMS or the ECM extract includes the native tissue components that preserve all or most of the ECM proteins. The TMS can also include scaffolding or supporting materials, such as decellularized native tissues.

In particular embodiments, the TMS does not include synthetic polymers. Synthetic polymers can be defined as polymers that are produced in a laboratory and/or do not occur naturally. In particular embodiments, the TMS and/or the compositions contain no added polymers, such as purified collagen and/or synthetic polymers.

In embodiments, the TMS includes a protein composition that is distinct from the protein composition of laminin-rich extracellular matrix (IrECM or Matrigel). For example, the protein content of the TMS can be <45% laminin, whereas the protein content of IrECM is >50% laminin. As another example, type I collagen can be the most abundant collagen type in the TMS, whereas type IV collagen is the most abundant collagen type in IrECM. In embodiments, the TMS can include proteins that are below the limit of detection in IrECM, as measured by mass spectrometry. In particular embodiments, the TMS can include one or more of periostin, tenascin-X, EMILIN-1, lumican, titin, perilipin ¼, elastin, dermatopontin, type V, VII, XI, XIV, XVI, and XXII collagen. In contrast, these proteins are below the limit of detection in IrECM, as measured by mass spectrometry.

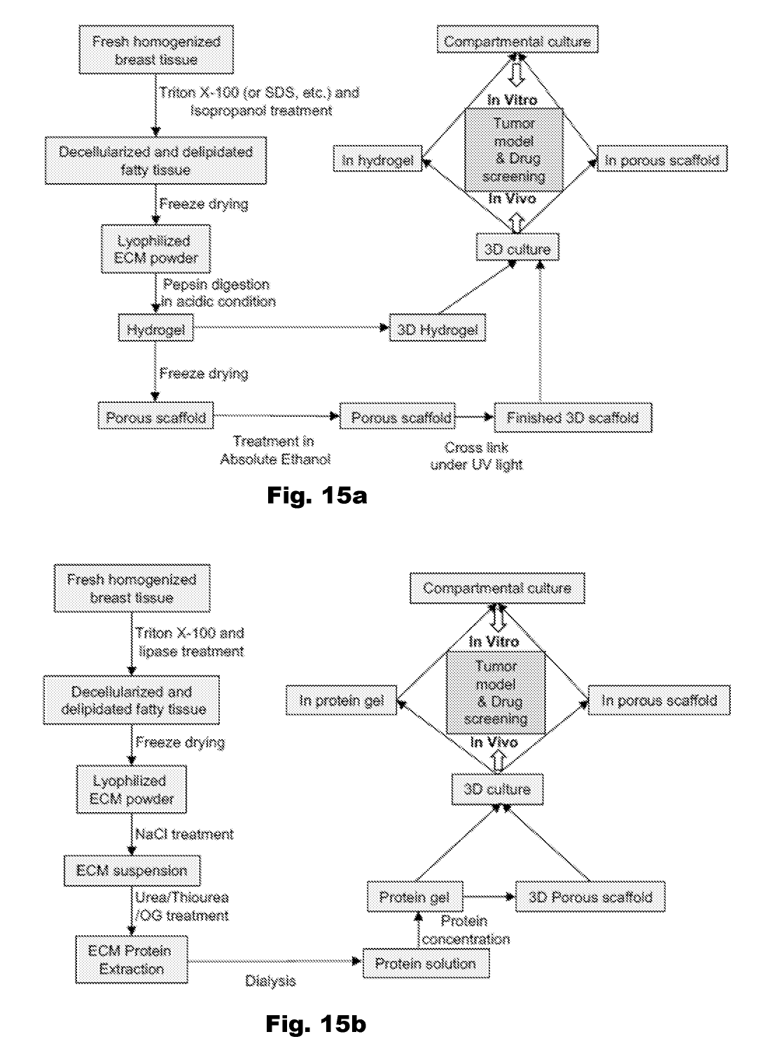

In particular embodiments, the protein content of a TMS or an ECM extract can include full-length ECM proteins, and/or can include fragments and/or peptides derived from the full-length ECM proteins. For example, proteins present in the TMS or ECM can be subjected to protease digest (e.g., pepsin), which can result in fragmentation of the full-length native proteins (see, e.g., FIG. 15a).

Examples of methods to homogenize tissue include grinding, douncing, blending, sonicating, bead-based disruption, nitrogen cavitation, pressurizing, macerating or pulverizing.

In embodiments, the TMS includes cross-linked molecules. In embodiments, cross-linked can refer to a bond that links one polymer chain to another polymer. Cross-linking of ECM molecules can contribute to the rigidity and structure of the ECM. In embodiments, native ECM present in tissues includes cross-links between various ECM molecules. In embodiments, production of TMS can include one or more cross-linking steps. In embodiments, the cross-linking step(s) can restore or enhance rigidity of the ECM structure present in the TMS. Examples of techniques for cross-linking include, but are not limited to, UV treatment or other high energy radiation, heat treatment, condensation, chemical reaction, free radical polymerization, aldehydes, crystallization, ionic interaction, protein interaction, and naturally-existing, native, cross-linker enzymes (e.g., tyrosinase, transglutaminase, sortase, subtilisin, laccase, peroxidase, lysyl oxidase, oxidoreductase, amine oxidase, etc.). Examples of other cross-linking agents includes but are not limited to non-native reagents, compounds, and metals.

In embodiments, the TMS or ECM extract is decellularized. Decellularized can refer to removal of cells from a tissue sample. In embodiments, a tissue sample can be decellularized in the process of making TMS by: treating the tissue sample with a surfactant, sonication, freeze/thaw, and/or sample agitation. Examples of surfactants, such as detergents, that can be used include but are not limited to sodium dodecyl sulfate and Triton X-100. In particular embodiments decellularization can include treatment with a surfactant and with alcohol. In embodiments, decellularization also results in the removal of DNA. In embodiments, decellularization results in the removal of about at least 90%, at least 95%, or at least 99% of the DNA, as compared to the DNA present in the tissue sample prior to decellularization (e.g., as measured by dry weight). In particular embodiments decellularization can be confirmed by hematoxylin and Eosin (H&E) staining, where no cell structure or nucleus can be identified in the decellularized sample.

In embodiments, the TMS or ECM extract is delipidated. Delipidated can refer to removal of lipids from a tissue sample to prepare a TMS. In particular embodiments, a sample can be delipidated by lipase or treatment with other enzymes, chemical methods (such as alcohol, urea, and sodium dodecyl sulfate treatment), and/or centrifugation. Lipase is an enzyme that can catalyze the hydrolysis of fats and oils. An example of a commercially available lipase is porcine pancreatic lipase, available from SIGMA-ALDRICH®.

In embodiments, producing TMS or ECM extract includes one or more dehydration steps. Dehydration refers to the drying of the TMS to remove liquids. Examples of techniques that can be used for dehydrating include but are not limited to vacuum drying, lyophilizing, dry air, air movement, heat or light drying, and other dehydrators. Dehydrating can help in generating pores in the TMS.

In embodiments, producing TMS or ECM extract includes one or more lyophilizing steps. Lyophilizing refers to the process of freezing and evaporating a solution under vacuum to produce a solid substance. Lyophilization, supercritical gas extraction, and/or chemical- or gas-based methods can help in generating pores in the TMS.

In embodiments, producing TMS can include converting an ECM extract powder into a liquid. In embodiments, an ECM extract powder can be dissolved in an acidic solution (e.g., a solution of at least 0.1% acetic acid and/or a pH of 6.6 or lower), which can further include a protease (e.g., pepsin). In particular embodiments, the acidic solution can be a 1% acetic acid solution, can have a pH of about 6.5, and can include pepsin. In particular embodiments, dissolving an ECM extract powder into a liquid can further include treatment with perchloric acid (e.g., 0.1%) in ethanol (e.g., 4%) and mixing for 4-6 hours.

In embodiments, producing TMS can include converting a liquid ECM extract into a hydrogel. In embodiments, a TMS that is dissolved in an acidic solution can be converted from a liquid to a hydrogel by neutralizing the pH of the solution. In particular embodiments, the pH can be neutralized by adding a basic solution (e.g., 0.1 N NaOH) to the liquid TMS until the pH is neutralized (to about pH 7.0) to form a hydrogel from the liquid ECM extract.

In embodiments, a porous TMS can refer to a dehydrated or dry TMS. The TMS can be produced in different shapes and sizes with desired porosity, which can be achieved by adjusting the amount of ECM used for preparing the TMS. Increasing the amount of ECM decreases the size of the pores, while decreasing the amount of ECM increases the size of the pores. The structural integrity of a TMS with a more dilute/highly porous ECM can be enhanced by treatment with tyrosinase, to further cross-link ECM proteins. The introduction of a naturally existing native cross-linker, such as tyrosinase, can provide controllable cross-linking of the ECM proteins which results in controllable stiffness or compliance.

A hydrogel TMS can refer to a TMS that is in a hydrogel form. A hydrogel is a type of colloid in which solid particles are dispersed in an aqueous liquid, and the solid particles form a rigid or semi-rigid network. Production of a hydrogel TMS can be similar to a porous TMS, except that the ECM is not subjected to a final dehydration or dehydration/cross-linking step, therefore retaining the hydrogel state of the TMS. Hydrogel TMS, however, can be subjected to dehydration or dehydration/cross-linking step to generate porous TMS.

In embodiments, the hydrogel TMS and porous TMS can be combined to form a multilayered TMS. In embodiments, a multilayered tissue matrix scaffold can include a porous TMS and one or more hydrogel TMS. In particular embodiments, a multilayered tissue matrix scaffold can include two layers of hydrogel tissue matrix scaffold, such that a first hydrogel TMS layer encases the porous TMS, and a second hydrogel TMS encases the outer surface of the first hydrogel TMS layer.

In particular embodiments, the TMS is a uniform TMS assembly support. A uniform TMS assembly support can refer to any TMS with multiple layers, wherein each layer is the same type of TMS (i.e., each layer is a hydrogel TMS or each layer is a porous TMS).

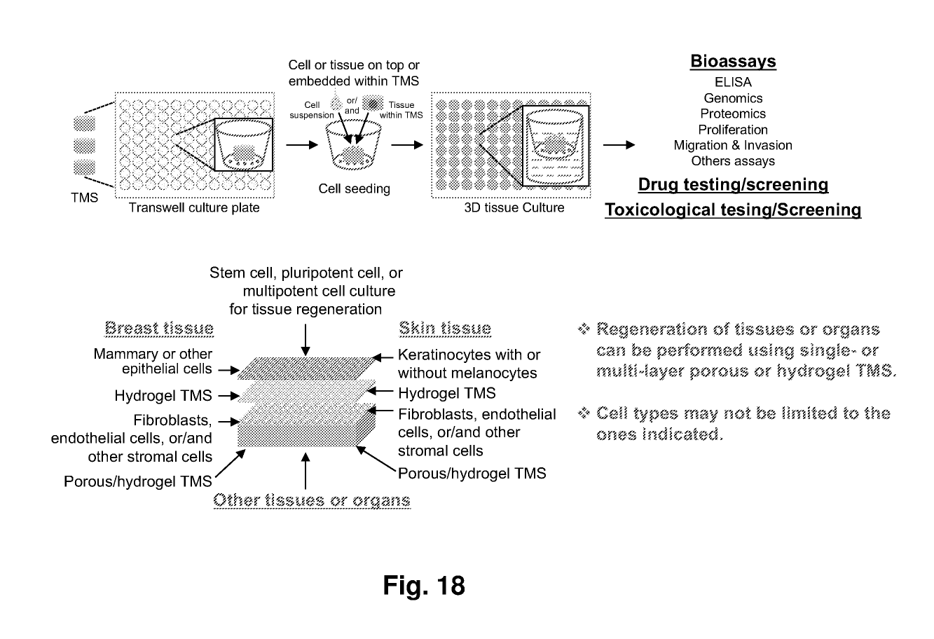

In particular embodiments, a multilayer, porous or hydrogel TMS can be used to model or generate breast tissue, skin tissue, and other tissues or organs and for research or other experiments, such as toxicological or drug screening, testing, or development. In embodiments, a multilayer, porous, or hydrogel TMS to model or generate breast tissue can include TMS in any forms of porous, non-porous, hydrogel forms, or the combination with or without inclusion of cells, such as, epithelial cells, fibroblasts and/or endothelial cells, stem cells, and other cells, such as macrophages, necessary for the action (FIG. 18). In embodiments, a multilayer or uniform TMS to model or generate skin tissue can include TMS in any forms of porous, non-porous, hydrogel forms, or the combination with or without inclusion of cells, such as stem cells, keratinocytes, fibroblasts, melanocytes, epithelial cells, and endothelial cells, necessary for the action (FIG. 18). In embodiments, a multilayer, porous, or hydrogel TMS to model or generate tissues other than breast or skin can include TMS in any forms of porous, non-porous, gel forms, or the combination with or without inclusion of cells, such as stem cells or/and other types of cells.

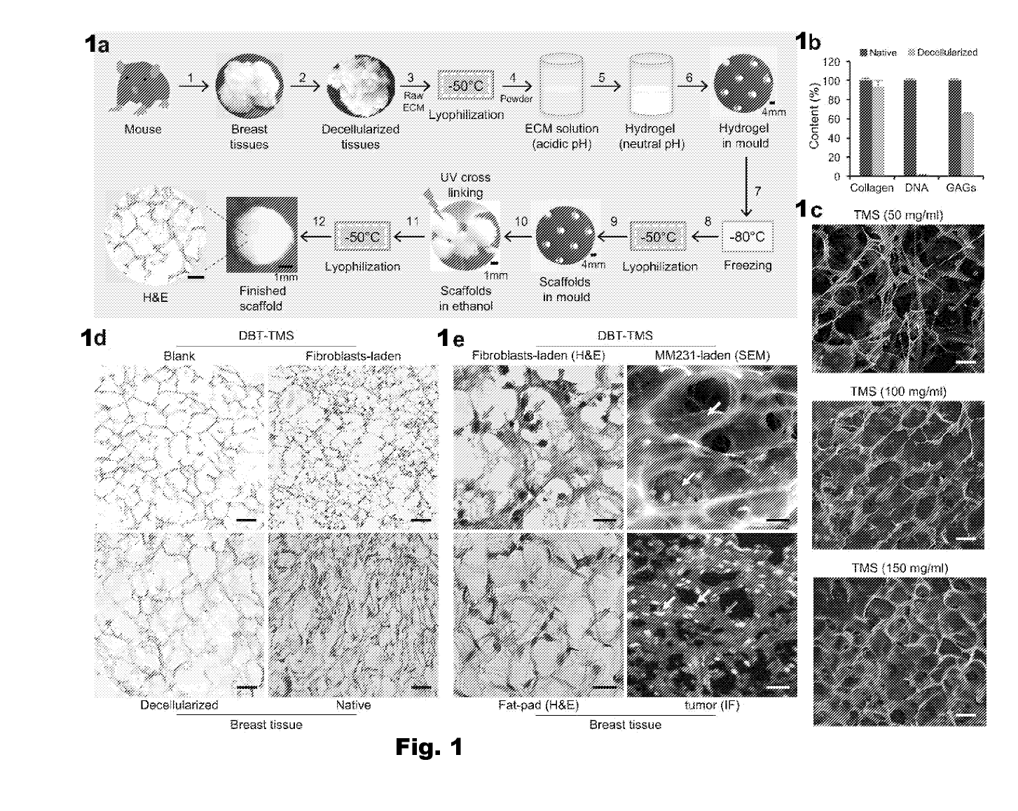

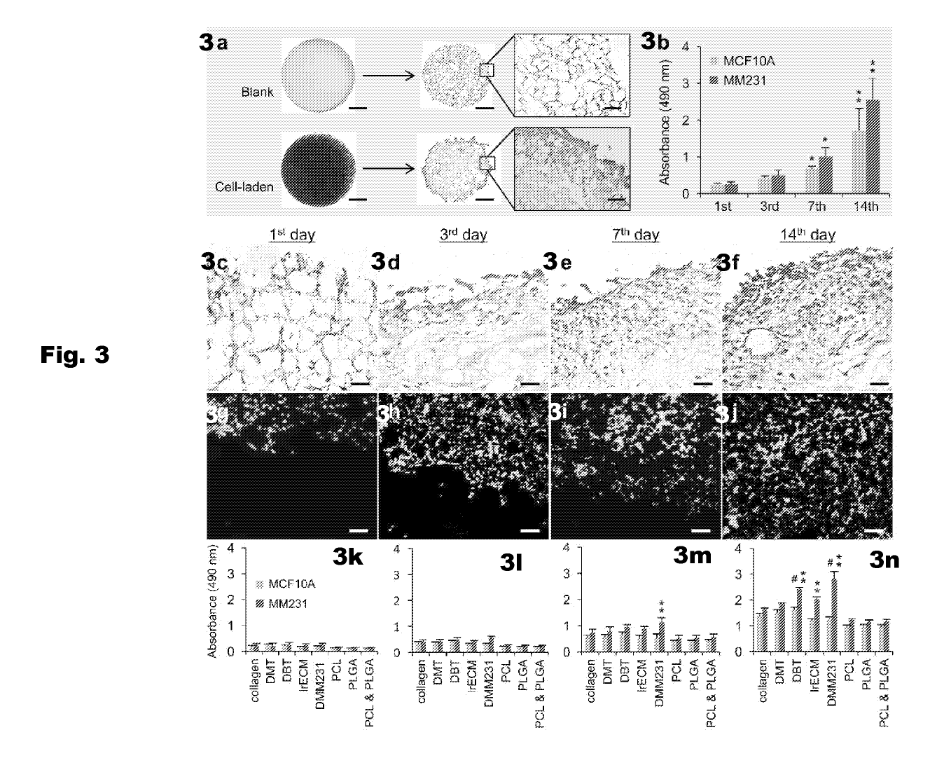

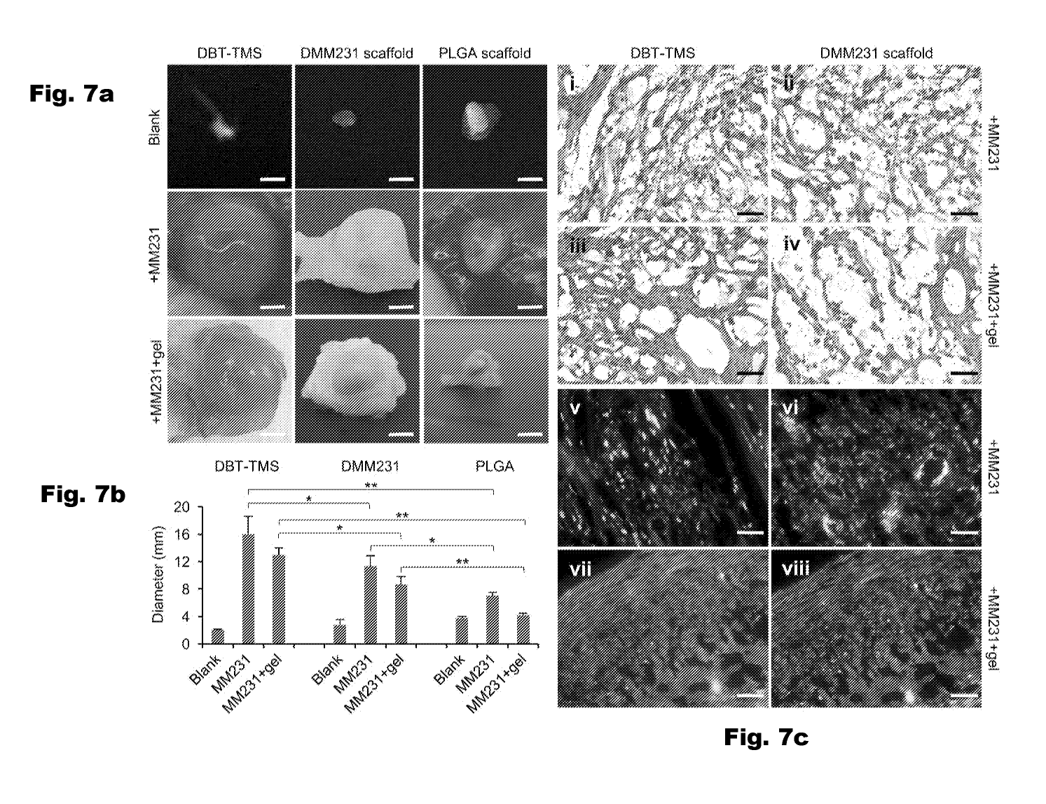

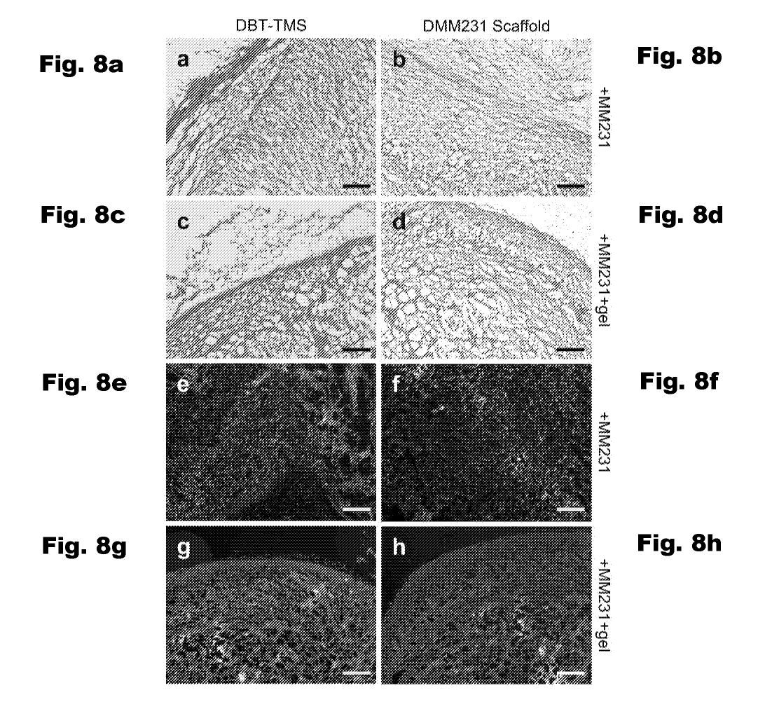

As shown in FIGS. 1d and 1 e, FIGS. 3a and 3c-j, FIG. 7, and FIG. 8, TMS has demonstrated its capability of supporting tissue-like structure formation in culture and tumor formation in mice. Depending on the type of cells seeded and grown into the TMS as well as the specific type of TMS or ECM source material used, breast or skin tissue and other tissues and organs can be constructed or produced by means of culturing, implantation, bioprinting, or the combination of the former techniques.

The construction or regeneration of breast or skin tissues using TMS can be achieved by growing different types of cells including pluripotent cells (e.g., induced pluripotent stem cells) or stem cells essential for the formation of the tissues on or within TMS (FIG. 18b). The TMS support for tissue regeneration can be in the forms of porous, hydrogel, and the combination of porous and hydrogel. The different forms of TMS can be applied as single compartment/layer or multi-compartment/multi-layer. Single type or multiple types of cells can be cultured using the TMS for tissue regeneration. The cells used for tissue regeneration can be cell lines or primary cells. In addition, TMS can be applied in the absence of cells to specific tissue area to form tissue support or to form initial scaffold for surrounding tissues growing into TMS to repair the local tissues.

In particular embodiments, cell types used to generate breast and skin tissues can include stem cells, pluripotent cells including induced pluripotent stem cells, and multipotent cells (e.g., precursor cells, and/or progenitor cells) that can differentiate into the cell types that are relevant to the tissue type that will be generated. For example, for regeneration of skin tissue, endothelial cells or endothelial progenitor cells can be used. As another example, for regeneration of breast or skin tissue, fibroblasts or fibroblast progenitors can be used.

In embodiments, one or more hydrogel TMS and/or one or more porous TMS can be combined into a multi-compartmental TMS, with or without separating semi-permeable membranes for tissue co-culture or other applications.

In particular embodiments, the TMS can be formed into a various shapes, including hemispherical, spherical, cubical, membrane or sheet shape.

In embodiments, a TMS (e.g., porous TMS, hydrogel TMS or multilayer TMS) can be shaped using a mould, punch, gravity, liquid, or mechanics and machineries. In particular embodiments, a porcelain or alternative material-based hemisphere, sphere, or other shapes of moulds can be used.

In embodiments, compositions including the delipidated or/and decellularized ECM extract can be in the form of a powder or a gel, or a combination of a powder and a gel. The powder, the gel, or the combination of powder and gel can be reconstituted to form a TMS. Reconstitution includes resuspending the powder, gel, or the combination of powder and gel in a solution, for example an acidic solution or buffer, to form a liquid with or without neutralizing the solution followed by cross-linking or natural polymerization to form a hydrogel TMS or a porous TMS.

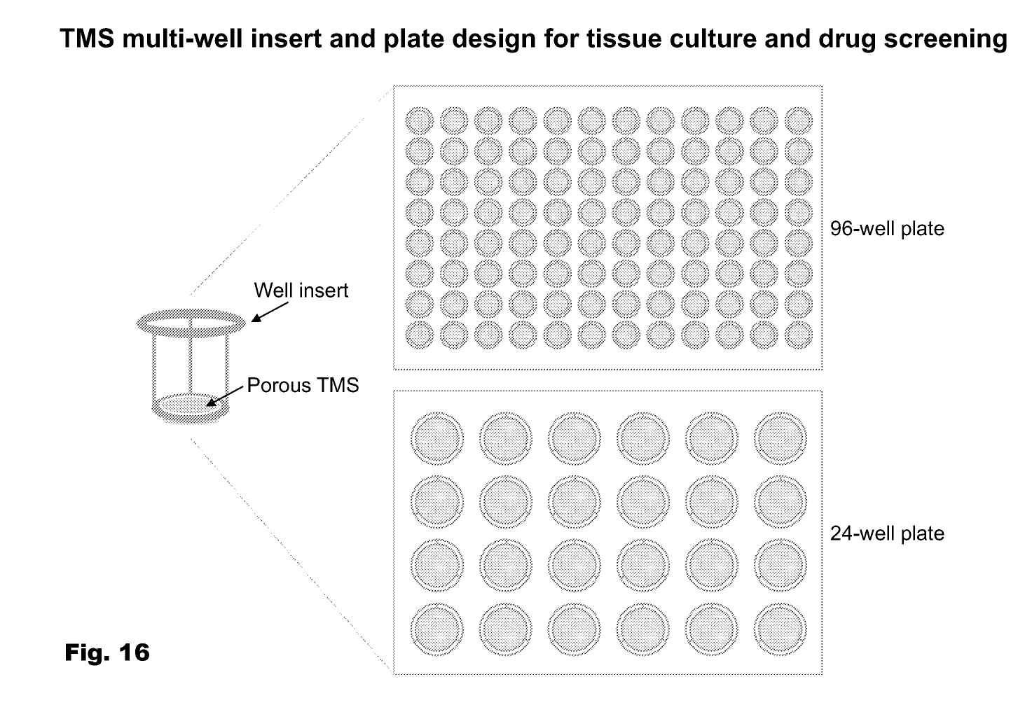

The TMS can be used for two- (in the forms of thin sheet or gel) or three-dimensional tissue culture. In embodiments, the TMS can be placed in single-well or multi-well inserts of culture places (FIG. 16 and FIG. 18). In embodiments, cells can be seeded on a TMS in the presence of culture media for two- or three-dimensional cell culture. In particular embodiments, a porous TMS can be used for three-dimensional cell culture, and cells suspended in growth media can be seeded and grown on the porous TMS. In particular embodiments, a hydrogel TMS can be used for three-dimensional cell culture, and cells suspended in growth media can be blended into the hydrogel TMS, or a piece of tissue can be embedded within the hydrogel TMS for further culture, experiments, or drug testing/screening. Blending cells into a hydrogel TMS can decrease the rigidity of the hydrogel. To increase rigidity after blending cells into the hydrogel, the hydrogel TMS can be further cross-linked. Increasing the rigidity of the hydrogel TMS can help the scaffold maintain a defined shape, which can be particularly useful, for example, when the hydrogel TMS becomes diluted by blending in a suspension of cells in growth media. In particular embodiments, a cross-linker enzyme (e.g., tyrosinase) can be used to cross-link the hydrogel TMS after the cells are added. In embodiments, a multilayer TMS can be used for cell culture, and cells can be grown on the surface or into the porous TMS layer(s) and embedded into the hydrogel TMS layer(s). In embodiments, a multilayer TMS can be useful for co-culturing multiple cell types or populations of the same cell type with different backgrounds of treatment or processing. For example, the hydrogel and porous layers can include distinct populations of cells, and/or can be made from ECM extracts derived from different tissue types.

In embodiments, a TMS can be used for various tissue culture research models. Research models that TMS can be used for include: cancer; diabetes; cardiovascular diseases; metabolic disorders; kidney diseases; lung diseases; liver diseases; gastrointestinal diseases; infectious diseases; neuronal disease; gynecological and obstetrics disease; pediatrics disease, immune disorders; tissue angiogenesis; cell migration; invasion and/or metastasis; tissue damage; biomarker testing and/or screening; toxicological testing and/or screening; drug testing and/or screening; and radiation testing and/or screening. The TMS can be used for understanding and monitoring the progression of a disease and for finding an appropriate therapy for treating the disease including determining the best therapeutic agent or/and regimen.

In embodiments, a TMS can be used to culture cells or serve as a support for xenograft, isograft, autograft, or allograft implantation in vivo. TMS is useful for xenograft, isograft, autograft, or allograft because compared to synthetic cell scaffolds, which contain synthetic polymers, TMS has improved biocompatibility, biodegradability, and less toxicity.

In embodiments, a TMS can be used for cell culture studies to assess the biomolecular profiles of cells grown in 2D or 3D culture. For example, cells can be cultured in a TMS under various growth conditions, and assessed by biomolecular profiling to determine the gene expression, protein, or small molecule profiles of the cultured cells or the secreted molecules from the cultured cells. Biomolecular profiling of cells cultured in TMS can be useful, for example, for studying cellular biology/biochemistry in the context of a native extracellular matrix. In particular embodiments, biomolecular profiling of cells can include genomic sequencing, proteomic profiling, metabolomic profiling, and/or immunostaining (e.g., immunofluorescence staining, immunohistochemistry, ELISA, and/or immunoblotting) as well as other molecular profiling methods or techniques. In particular embodiments, the biomolecular profiling can be performed for cross sections of cell-laden or tissue-carrying TMS culture samples (e.g., tumoroid cross sections or patient tissue/cell-embedded TMS cross sections), whole cell-laden TMS culture samples (e.g., an intact tumoroid or patient tissue/cell-embedded TMS), culture supernatant, and/or cell isolated from the culture.

In embodiments, a TMS culture system can be used to screen candidate drugs such as small molecules or biologics for treating various diseases including various cancers, diabetes, metabolic disorders, cardiovascular diseases, kidney diseases, lung diseases, liver diseases, gastrointestinal diseases, infectious diseases, neuronal disease, gynecological and obstetrics disease, pediatrics disease, and immune disorders. A TMS culture system can be useful for testing/screening candidate drugs or the toxicities of the drugs for their efficacies towards tumors, cancers, or other diseases. As an example, a 3D scaffold that includes a native tissue ECM can more accurately model an in vivo tumor microenvironment than a 2D culture system, a synthetic polymer scaffold, or a single ECM component scaffold. Examples of small molecule candidate drugs that can be screened include, but are not limited to, tamoxifen, paclitaxel, raloxifene, methotrexate, docetaxel, doxorubicin, 5-fluorouracil, trastuzumab, pertuzumab, cyclophosphamide, doxorubicin, epirubicin, cisplatin, pembrolizumab, olaratumab, sorafenib tosylate, carboplatin, vorinostat, rituximab, bevacizumab, cetuximab and imatinib for the treatment of cancer.

Tamoxifen is a cancer drug used for both early and advanced estrogen receptor (ER)-positive (ER+) breast cancer, and is commercially available as Soltamox® and Novadex®. After administration, tamoxifen is catabolized to the active metabolite (Z)-4-hydroxytamoxifen. In particular embodiments, screening can include treating cells in a TMS with (Z)-4-hydroxytamoxifen. Paclitaxel is a chemotherapy agent used for a variety of cancers including ovarian cancer, breast cancer, lung cancer, Kaposi sarcoma, cervical cancer, and pancreatic cancer. Paclitaxel is commercially available as Taxol®.

Other candidate drugs can be screened for the treatment of different kinds of cancers, diabetes, metabolic disorders, cardiovascular diseases, kidney diseases, lung diseases, liver diseases, gastrointestinal diseases, infectious diseases, neuronal disease, gynecological and obstetrics disease, pediatrics disease, and immune disorders. The candidate drug could be a well-known drug for a certain indication, and it could be screened using the TMS system for a different indication. The candidate drug may be a novel drug and could be screened for treatment of a specific disease. The candidate drug may be a compound or other molecule that does not yet have a known therapeutic use.

In embodiments, screening candidate drugs can include evaluating the treated cells to determine anti-cancer or anti-tumor efficacy, or the efficacy to treat other diseases. Examples of methods of evaluating treated cells include biomolecular profiling of the cells and assessing: cell proliferation, cell survival, cytotoxicity, tumor/tumoroid size, and/or cell metabolism. Examples of assays for assessing cell proliferation, survival and/or cytotoxicity, migration, invasion and/or metastasis include: CCK-8, Live/Dead Cell Staining, Trypan blue, TUNEL, and immunostaining and microscopy (for cell migration and/or invasion). Tumor/tumoroid size can be assessed, for example, by microscopy and analysis of images with software such as ImageJ. Examples of assays for assessing cell metabolism include lactate dehydrogenase, MTT, mass spectrometry, nuclear magnetic resonance spectroscopy, and AGILENT SEAHORSE® metabolic profiling.

In embodiments anti-cancer or anti-tumor efficacy can refer to a statistically significant decrease in cell proliferation, survival, metabolism and/or tumor/tumoroid size in the presence of a candidate drug, as compared to a negative control or the absence of the candidate drug. In particular embodiments, anti-cancer or anti-tumor efficacy can refer to a statistically significant increase in cancer cell cytotoxicity in the presence of the candidate drug, as compared to a negative control or the absence of the candidate drug.

In embodiments, a TMS culture system can be used to form a tumoroid. A tumoroid can refer to an aggregate of cancer cells and/or tumor-derived cells. A tumoroid can refer to a tumor-like structure or appearance that resembles a tumor. In embodiments, a tumoroid can be implanted in vivo to induce production or growth of a tumor in a research animal. In particular embodiments, implanting can include injecting the tumoroid into an animal with a syringe, or surgically inserting the tumoroid at an excision site.

In embodiments, production or growth of a tumor in vivo can be evaluated by (i) detecting and measuring a tumor that is visible at the surface of an animal's skin, (ii) imaging techniques such as MRI or Positron Emission Tomography and Computed Tomography PET/CT, and/or (iii) removing and assessing the tumor and/or tissue surrounding the tumor. Assessing the tumor and/or tissue surrounding the tumor can include, for example, histological or/and pathological examination of tissue cross sections.

In embodiments, forming a tumoroid in a TMS culture system can lead to more rapid tumor formation/production when implanted in vivo, as compared to a tumor formed from the support of a synthetic polymer scaffold or injection of cancer cells in vivo. In particular embodiments, the rapid tumor production can be evidenced by a larger tumor diameter, as measured at different time points after implantation (e.g., four weeks after implantation).

Also disclosed herein are kits for producing TMS from ECM extract, for culturing cells in TMS, and/or for screening drug candidates in TMS. In embodiments, the kits contain ECM extract or TMS, one or more containers including one or more TMS or reagents for producing TMS, as described herein. The kits can also include instructions for using the kits and/or a notice in the form prescribed by a governmental agency regulating the manufacture, use, or sale of pharmaceuticals or biological products, which notice reflects approval by the agency of manufacture, use, or sale for human administration.

In embodiments the kits include plates/dishes for culturing cells in the TMS, such as 24-well plates or 96-well plates. In embodiments, the kits include single-well inserts and/or multi-well inserts that fit into wells of multi-well plates, and include a porous TMS at the bottom of the insert. The single-well or multi-well inserts can carry round, square, or other shapes of porous or hydrogel TMS that are uniquely designed for tissue culture or drug screening using multi-well culture plates. The multi-well inserts can be individual or panelized. In particular embodiments, the kits include reagents for evaluating the various types of cells, such as reagents for a cell proliferation assay. In particular embodiments, the dishes, plates, or multi-well inserts can be pre-loaded with TMS.

In embodiments, kits for producing TMS from an ECM extract include moulds for shaping the TMS. In particular embodiments, the moulds can be hemispherical-, spherical-, cubical-shaped, or other shapes.

### Exemplary Embodiments

1. A composition including tissue-derived extracellular matrix (ECM) extract, wherein the extract includes a protein content of less than about 45% laminin, and wherein the extract is decellularized and delipidated.
2. The composition of embodiment 1, wherein the extract is in a powder form.
3. The composition of embodiment 1 or 2, wherein the less than about 45% laminin is less than 40%, less than 30%, less than 20%, less than 10%, or less than 5% laminin,
4. The composition of any one of embodiments 1-3, wherein the extract does not include synthetic polymers.
5. The composition of any one of embodiments 1-4, wherein the extract is derived from a mammal.
6. The composition of embodiment 5 wherein the mammal is mouse, pig, human, cow non-human primate.
7. The composition of any one of embodiments 1-6 wherein the extract is derived from one or more of mammary tissue, muscle tissue, adipose tissue, skin tissue, heart tissue, liver tissue, brain tissue and/or lung tissue.
8. A hydrogel tissue matrix scaffold (TMS) including a hydrogel of a composition of any one of embodiments 1 or 3-7.
9. A porous tissue matrix scaffold (TMS) including a decellularized, delipidated and dehydrated tissue-derived extracellular matrix (ECM) extract.
10. The TMS of embodiment 9, wherein the TMS includes a protein content of less than about 45% laminin.
11. The TMS of embodiment 10, wherein the less than about 45% laminin is less than 40%, less than 30%, less than 20%, less than 10%, or less than 5% laminin.
12. The TMS of any one of embodiments 9-11, wherein the TMS does not include artificially added polymers.
13. The TMS of any one of embodiment 9-12, wherein the extract is derived from a mammal.
14. The TMS of any one of embodiments 9-13, wherein the mammal is mouse, pig, human, cow, or non-human primate.
15. The TMS of any one of embodiments 9-14, wherein the extract is derived from one or more of mammary tissue, muscle tissue, adipose tissue, skin tissue, heart tissue, liver tissue, brain tissue and/or lung tissue.
16. The TMS of any one of embodiments 9-15, wherein the TMS is hemispherical, spherical, cubical, a sheet, or a membrane.
17. A multilayered TMS including (i) a porous TMS of embodiment 9; and (ii) one or more hydrogel TMS of embodiment 10.
18. The multilayered TMS of embodiment 17, wherein a first hydrogel TMS layer encases the porous TMS, and wherein a second hydrogel TMS encases the outer surface of the first hydrogel TMS layer.
19. A method of producing a TMS including:

obtaining a sample derived from a fresh, homogenized tissue;

decellularizing and delipidating the tissue;

lyophilizing the decellularized and delipidated tissue to obtain a powder;

suspending the powder in a buffer to form the hydrogel; and

allowing the hydrogel to solidify;

thereby producing a TMS.
20. The method of embodiment 19 wherein allowing the hydrogel to solidify includes (i) solidifying the hydrogel in a hemispherical, spherical, cubical mould, or (ii) coating a surface with the hydrogel and allowing the hydrogel to solidify.
21. The method of embodiment 19 or 20 further including:

dehydrating and cross-linking the solidified hydrogel to produce a porous TMS;

thereby producing a porous TMS.
22. A method of producing a TMS including;

obtaining a sample derived from a fresh, homogenized tissue;

decellularizing and delipidating the tissue;

lyophilizing the decellularized and delipidated tissue to obtain a powder;

suspending the powder in a buffer to form a liquid;

extracting the protein from the liquid to form a liquid ECM extract; and

concentrating the liquid ECM extract to form a hydrogel;

thereby producing a TMS.
23. The method of embodiment 22 further including:

dehydrating the hydrogel to form a porous solid;

thereby producing a porous TMS.
24. The method of any one of embodiments 19-23, wherein the decellularizing includes treating the tissue with a surfactant.
25. The method of any one of embodiments 19-24, wherein delipidating includes treating the tissue with lipase or centrifuging the sample to remove lipids.
26. A method of preparing a three-dimensional cell culture, the method including seeding cells or tissue on or into the TMS of any one of embodiments 8-18 and allowing the cells to proliferate.
27. The method of embodiment 26, wherein the seeding includes inserting cells suspended in growth media or tissue into the TMS.
28. The method of embodiment 27, the method further including pre-conditioning the TMS by submerging the TMS in growth media before inserting the cells suspended in growth media into the TMS.
29. The method of embodiment 26 or 27, wherein the TMS is a hydrogel TMS and the seeding includes (i) blending the cells suspended in growth media into the hydrogel TMS; or (ii) growing the cells on the surface of the polymerized hydrogel TMS.
30. The method of embodiment 29, the method further including cross-linking the hydrogel TMS after seeding the cells.
31. The method of embodiment 30, wherein the cross-linking includes treating with a cross-linking enzyme.
32. The method of embodiment 31 wherein the cross-linking enzyme includes tyrosinase
33. The method of any one of embodiments 26-32, wherein the three-dimensional cell culture is a research and/or a testing/screening model.
34. The method of embodiment 33, wherein the research model and/or testing/screening model is used to model one or more of: cancer; metabolic disorders, diabetes; cardiovascular; metabolic disorders; kidney diseases; lung diseases; liver diseases; gastrointestinal diseases; infectious and immune disorders; neuronal disease; gynecological and obstetrics disease; pediatrics disease; tissue angiogenesis; cell migration, invasion and/or metastasis; tissue damage; biomarker testing and/or screening; toxicological testing and/or screening; small molecule/particle testing and/or screening; and radiation testing and/or screening.
35. The method of any one of embodiments 26-34 wherein the three-dimensional cell culture is used to prepare cells or tissue for implantation/transplantation, research and/or preclinical/clinical applications,
   wherein the cells or tissues are skin or mammary cells or tissues.
36. A method of generating tissue including

seeding a multilayer TMS with cells;

adding growth medium to the seeded multilayer TMS; and

allowing the cells to proliferate on the multilayer TMS, thereby generating tissue.
37. The method of embodiment 36, wherein the cells include at least two different types of cells.
38. The method of embodiment 37, wherein the cells include (i) keratinocytes and fibroblasts; or (ii) epithelial cells, fibroblasts, and/or endothelial cells.
39. The method of embodiment 37 or 38, wherein the different types of cells are seeded on different layers of the TMS,
40. The method of embodiment 38 or 39, wherein the keratinocytes are seeded on top of a first TMS layer, and wherein the fibroblasts are seeded onto a second TMS layer that is layered below the first TMS layer.
41. The method of embodiment 38 or 39, wherein the epithelial cells are seeded on top of a first TMS layer and wherein the fibroblasts and/or endothelial cells are seeded onto a second TMS layer that is below the first TMS layer;
42. The method of embodiments 40 or 41, wherein the first TMS layer is a hydrogel TMS and the second TMS layer is a porous TMS.
43. The method of any one of embodiments 36-40 or 42, wherein the tissue is skin tissue.
44. The method of any one of embodiments 36-39, 41, or 42, wherein the tissue is breast tissue.
45. A method of preparing a tumoroid for in vivo implant, the method including

seeding the TMS of any one of embodiments 8-18 with cancer cells,

preculturing the cancer cells in the TMS in vitro to allow a tumoroid to form.
46. The method of embodiment 45 further including seeding the TMS with a second population of cells.
47. The method of embodiment 46 wherein the second population of cells includes fibroblasts.
48. The method of any one of embodiments 45-47, the method further including implanting the tumoroid in an animal and allowing the tumoroid to proliferate.
49. The method of embodiment 48, wherein proliferation of the tumoroid in the animal is faster than proliferation of a tumoroid formed from cancer cells seeded on or within a synthetic polymer scaffold and implanted in an animal.
50. The method of any one of embodiments 45-49, wherein time-period of preculturing to allow the tumoroid to form is shorter than a time-period of preculturing to allow a tumoroid to form from cancer cells seeded in a synthetic polymer scaffold.
51. A method of testing one or more candidate drugs for anti-tumor efficacy, said method including seeding the TMS of any one of embodiments 8-18 with cancer cells, treating the tumoroid that forms from the cancer cells with the one or more candidate drugs, and evaluating the cancer cells to determine anti-tumor efficacy of the one or more candidate drugs.
52. The method of embodiment 51, wherein evaluating the cancer cells includes assessing one or more of: cell proliferation, cell survival, migration, invasion, metastasis, cytotoxicity, tumor or tumoroid size, and/or cell metabolism.
53. The method of embodiment 51 or 52, wherein the one or more candidate drugs are tamoxifen or paclitaxel.
54. A method of preparing the hydrogel TMS of embodiment 8 from an ECM extract including dissolving the ECM extract in a buffer to form a hydrogel, thereby preparing a hydrogel TMS from an ECM extract.
55. A method of preparing the porous TMS of embodiment 9 from an ECM extract including dissolving the ECM extract in a buffer to form a hydrogel, thereby preparing a porous TMS from an ECM extract.
56. A kit for producing a TMS including the composition of any one of embodiments 1-7, and reagents and/or instructions for producing a TMS from the composition.
57. The kit of embodiment 56, the kit further including (i) a mould for shaping the TMS or (ii) an object for coating with the TMS.
58. The kit of embodiment 57, wherein the mould is a hemispherical, spherical, or cubical mould.
59. The kit of embodiment 57, wherein the object for coating with the TMS is a coverslip or a microscope slide.
60. A kit for drug screening, the kit including the TMS of any one of embodiments 8-18, and one or more of: plates or dishes for culturing cells in the tissue matrix scaffolds; reagents for evaluating the cells, and instructions for using the TMS for drug screening.
61. The kit of embodiment 60, wherein the plates or dishes are coated or covered with the TMS.
62. The kit of embodiment 60, further including single-well or multi-well inserts containing the TMS.
63. A kit for culturing cells in a tissue matrix scaffold, the kit including a TMS of any one of embodiments 8-18, and instructions for seeding the TMS with cells.
64. The kit of embodiment 63, wherein the plates or dishes are coated or covered with the TMS.
65. The kit of embodiment 63, further including single-well or multi-well inserts containing the TMS.
66. A kit for generating tissue, the kit including:
   a porous TMS;
   (i) an ECM extract of any of embodiment 1-7 and instructions for producing a hydrogel TMS from the ECM extract, or (ii) a hydrogel TMS; and
   instructions for seeding cells in or on the porous TMS and the hydrogel TMS.
67. A kit of embodiment 66 further including dishes or plates, wherein the dishes or plates are coated with the porous TMS.
68. A kit of embodiment 67 further including instructions for coating the porous TMS with a layer of hydrogel TMS.

### EXAMPLES

Introduction. Cancer cells living in human tissues have contacts with ECM at all directions, and interact with other cells of the same or different types in their vicinity. The biological activities of the cells not only are passively affected by the physicochemical changes of the ECM, but also actively modify the ECM by applying expansion forces and by secreting enzymes that facilitate the survival and spread of the cancer cells. It is conceivable that the tumor locus is a spatial and temporal microenvironment undergoing consistent remodeling with molecular relays at extra-, inter- and intracellular levels. With the increasing understanding about the microenvironment of tumor tissues and the signaling cue-oriented cell phenotypes, many tumor biomedical studies inspecting cell signaling, gene and small molecule expression, and drug sensitivities have adopted different 3D tissue culture models (1). Overall, cancer cells grown in 3D cultures display different morphologies, motilities, proliferation capacities (2, 3), and higher resistance to anticancer drugs (4, 5) compared to those on flat surfaces.

Cell spheroids and scaffolds are the most popular 3D tissue culture models currently used in the field. Spheroids are clusters of cells that are often applied to mimic breast acinar structures, model epithelial cancer formation, and assess endothelial cell angiogenesis (2, 6, 7). Yet, they are not considered as ideal models for cancer studies because of the inconsistencies in their formation that varies with cell types (6), the challenges in handling, and the controversial biological relevance (8). Scaffolds exist in hydrogel or porous forms, and are made from either natural materials or synthetic polymers as previously described (1, 9, 10). Hydrogels prepared from specific component(s) of ECM, such as collagen and fibronectin, non-mammalian biomaterial alginate, and hydrophilic synthetic polymers, such as Poly(Ethylene Glycol) (PEG), have been used in various 3D cell cultures. However, the lack of the necessary tissue ECM components limits the applications of these types of hydrogels in the studies of mammalian cell biology, and compromises the reliability of the related data for the interpretations of human pathophysiological conditions. On the other hand, the broadly used laminin-rich ECM (IrECM) hydrogel or its equivalent Matrigel generated from the basement membrane (BM) extracts of the Engelbreth-Holm-Swarm (EHS) mouse sarcoma contains more complex ECM proteins and growth factors (11-13). Since the tumor and normal tissue ECM are different and the ECM components are critical for the expression of specific cell surface receptors (14), the tumor-derived laminin- and collagen IV-rich hydrogel may not be appropriate for the experiments involving culturing normal cells, especially normal stromal cells, or irrelevant cancer cells. Consistent with this notion, the growth factors contained in the Matrigel was found influencing cellular activities (13), and the breast normal epithelial and cancer cells displayed different phenotypes in IrECM culture, with distinct capacities in depositing their endogenous BM-like material (15). So far, the predominant usage of the IrECM hydrogel stays in its gel formats for coating culture vessels, embedding cells, or injection into animals as a carrier for the testing agents. IrECM-based solid porous scaffolds have not been established.

The current porous scaffolds are mostly synthesized using polymers, such as polycaprolactone (PCL) and poly(lactic-co-glycolic) acid (PLGA), and generally used for tissue engineering studies although there is an increased implementation of synthetic polymer-based scaffolds in 3D cell cultures (1). Overall, the hydrophobic and non-biological natures of the polymers, in addition to the adverse effects from their degradation products, hamper the biomedical applications of these types of scaffolds. Decellularized native tissues have been considered as ideal scaffolding materials for bioengineering and biomedical studies (16). However, the development of tissue-derived scaffold models is lagging. The decellularized tissues have only been used as hydrogel to coat plates, mixed with synthetic polymers in tissue engineering studies (17), in the “wet” native form for 3D cell culture (18), or as cryoprotected matrix for transplantation (19). Clearly, a more advanced, user-friendly and biologically relevant tissue ECM-based culture model is needed for more in-depth mechanistic and therapeutic studies of human pathophysiological conditions.

Generally, an elastic hydrogel scaffold is formed upon polymerization or cross-linking of the gelatinous suspension of monomeric or polymeric scaffolding materials. The sol-gel transition is typically induced by changes in pH, temperature, ionic composition, or illumination (20-22). For 3D tissue culture with hydrogel, normally the cells of interest are mixed in before polymerization. Recently, peptide hydrogel derived from decellularized porcine ECM has been printed into a polymeric PCL scaffold to produce tissue analogues (23). Yet, the scaffolding process requires large amounts of tissues to prepare the pro-gel, robotic instruments to cast the product, and involve the use of synthetic polymers to serve as structural scaffold. These requirements pose a challenge to most research laboratories' intent on conducting mechanistic and therapeutic studies due to the high cost of such instruments and the difficulties to obtain large amount of animal tissues. Importantly, a natural ECM-derived porous scaffold without synthetic polymer supports has not been reported. Therefore, there is a need for an alternative approach to meet specific design requirements aimed at mimicking the native microenvironment of human tissues. A scaffold having a porous structure enables cells to obtain nutrients, growth factors, and other biomolecules for their survival, growth, proliferation and migration. Additionally, with co-culture of stromal cells that can produce the ECM proteins, cancer cells grown in a porous scaffold can acquire de novo ECM microenvironment that best serves the cells' needs.

### Example 1: Fabrication and Characterization of the TMS

Materials and Methods. Reagents. Sodium dodecyl sulphate (SDS), sodium bicarbonate, and Fetal bovine serum (FBS) were purchased from Thermo Fisher Scientific. Triton X-100, perchloric acid, Sigmacote®, chloroform, absolute ethanol, xylene, Organo/Limonene mounting media, hematoxylin and eosin solutions, pepsin, tyrosinase, Cell Counting Kit-8 (CCK-8), Hydroxyproline Assay Kit for collagen content measurement, collagen from bovine skin, IrECM from Engelbreth-Holm-Swarm mouse sarcoma, PLGA, and PCL were purchased from Sigma-Aldrich. Live/Dead Cell Staining Kit II was purchased from PromoKine (PK-CA707-30002).

Cells and culture media. The MCF10A, MDA-MB-231, T47D, BT474 and NIH/3T3 cells were purchased from American Type Culture Collection (ATCC). The GM637 cell line was a gift from Dr. Richard Anderson at the University of Wisconsin-Madison. The cell culture media 1×DMEM/F12 50/50 (for MCF10A cells; supplemented with 5% horse serum, 20 ng/ml EGF, 0.5 μg/ml hydrocortisone, 100 ng/ml cholera toxin, 10 μg/ml insulin, 1% Penicillin-Streptomycin) and 1×DMEM (for the cancer cells; supplemented with 10% FBS and 1% Penicillin-Streptomycin) were purchased from Mediatech, Inc., USA.

Antibodies. Primary rabbit antibody for HER2 (#2165) and mouse antibody for Ki-67 (#9449) were purchased from Cell Signaling Technology. Alexa Fluor® dye-conjugated anti-rabbit and anti-mouse secondary antibodies were purchased from Thermo Fisher Scientific.

Anticancer drugs. (Z)-4-Hydroxytamoxifen (HT) and Paclitaxel (Taxol) were purchased from Abcam (#ab1419430) and Sigma-Aldrich (#T19120), respectively.

Microscopy. Zeiss Imager M2 upright epifluorescence microscopy at WSU Microscopy Core facility was used for both bright field and fluorescence imaging. FEI Quanta 200F scanning electron microscope (SEM) at WSU Franceschi Microscopy & Imaging Center was used for SEM imaging.

TMS fabrication from animal tissues. Mammary or muscle tissues were isolated from NOD/SCID mice (8-12 weeks old). Decellularization of the tissues was performed following an improved protocol based on previous reports (17, 28, 59). Briefly, the collected tissues were sliced into small pieces, centrifuged to remove fatty oil, and washed in 1×PBS for three times. SDS solution (0.5% for the mammary tissues, and 1% for the muscle tissues) in 1×PBS was used to decellularize the tissues at room temperature (RT) for 48 hours (replacing the solution every 10-12 hours). The processed mammary tissues were treated with isopropyl alcohol for 48 hours (replacing the solution every 10-12 hours), and the muscle tissues were treated with 1% Triton X-100 solution in PBS for 30-60 minutes. After several rounds of washing in 1×PBS, the decellularized breast or muscle tissues (the ECM) were lyophilized in a freeze-dryer (115 mT of vacuum drying rate, MillrockTechnology) at −50° C. for 24 to 48 hours depending on the volume of the samples. The DBT and DMT were then ground separately in liquid nitrogen to make powder forms of the ECM. The required amount of the decellularized ECM was digested in acidic pepsin solution (10 mg pepsin in 1 ml of 1% acetic acid solution in 1×PBS) until completely dissolved at RT or 37° C. Perchloric acid (0.1%) in 4% of ethanol was added into the acidic gel-like solution and mixed for 4-6 hours. The solution was neutralized using 0.1 N NaOH solution to form hydrogel at 4° C. and stored until use.

Porcelain files containing hemispherical moulds at desired diameters (2, 3, or 4 mm) were generated, and coated with the hydrophobic microscopically thin film of chlorinated organopolysiloxane colorless solution (Sigmacote®) according to the manufacturer's instructions. The prepared hydrogel at equal amount was slowly added into the wells of the moulds to make spherical structures at 4° C., and transferred to −80° C. for 1-4 hours to preserve the shape of the pre-scaffolds. When solidified, the pre-scaffolds were lyophilized at −50° C. for 24 hours. After lyophilization, the scaffolds were dipped into absolute ethanol, and exposed to UV light (20000 KJ) for 30 seconds to cross-link the ECM, followed by another round of lyophilization for 3-6 hours. The finished porous scaffolds were collected and kept dry in 4° C. for further experiments.

TMS fabrication from large scale animal tissues (summarized in FIG. 15b).

### Example 1A

Tissue collection and decellularization. The fresh tissues or organs from pigs were collected aseptically from a local slaughter house, where the purpose of using the designated tissues was informed, and transferred to the lab as soon as possible in ice bag. For TMS production using pig breast tissues, the tissues were sliced into small pieces and homogenized in sterilized and ice-cooled deionized distilled water. The homogenized breast tissues were centrifuged to remove the fat at a speed of 10,000 rpm for 30 minutes at 42° C. (Pig fat is relatively sticky and melts only above 37° C.). Centrifugation process was repeated to remove the fat until there was visible of oil droplets on the surface. The supernatant was discarded, and 0.1% Triton X-100 was added in sufficient amount to the homogenized tissues (at least 10 times more than the homogenized tissue volume) and mixed for 12 hours at room temperature. The process was repeated one more time. The mixture was centrifuged as described above, and sediment of decellularized extracellular matrix (ECM) was transferred into another tube containing 0.1% Triton X-100, protease inhibitor cocktail tablet (one tablet/10 ml), and lipase (1 mg/1 gm of ECM). The volume was adjusted according to the ECM amount. The tube containing the sample was incubated and rotated at 37° C. for 12 hours, followed by several rounds of washing with deionized distilled water and centrifugation to ensure complete removal of Triton X-100, protease inhibitors, and lipase from the sample. The final ECM was lyophilized.

### Example 1B

Extraction of ECM proteins. The lyophilized ECM was pulverized by grinding in liquid nitrogen, followed by treating the ECM powder twice with 3.4 M NaCl buffer (NaCl—99.25 g, 2M Tris Base—6.25 ml, EDTA 0.75 g and distilled water to 500 ml, final pH 7.4) for 15 minutes at 4° C. The ECM was pelleted by centrifugation and homogenized in 2 M urea buffer (Urea 60 g, Tris Base 3.025 g, NaCl 4.5 g, distilled water to 500 ml, pH 7.4) at 4° C. overnight. The sample was then centrifuged at 13,000 rpm for 30 minutes. The supernatant was collected and kept on ice. Homogenization of the ECM sediment was followed using 4M and 6M urea buffer, respectively, and the supernatant from each extraction was collected and stored as described above. The remaining insoluble ECM was treated with increasing concentration of Urea/Thiourea (6 M/0.5 M; 6 M/2 M; 7 M/0.5 M; and 7 M/2 M) for 12 hours at 4° C., and supernatant collected as before. The insoluble ECM sediment was further homogenized with 8M urea, and then with 2% of n-octyl B-D-glucopyranoside (OG) overnight at 4° C. Again, the supernatant was collected as before after centrifuged at 13,000 rpm for 30 minutes. The urea concentrations of the different batches of the supernatants were brought to 2M. Then the tissue ECM protein extracts were pooled together and dialyzed in cold TBS (Tris Base 6.05 g, NaCl 9.0 g, pH 7.4, total volume of 1 L with 5 ml of chloroform) for at least 2 hours. Dialysis was repeated twice in cold TBS without chloroform for 12 hours. Further dialysis with serum free 1×DMEM medium is optional. The sterile ECM solution was then concentrated using polyethylene glycol (PEG) and stored at −20° C. to −80° C. for future use.

Characterization of the decellularized animal tissue ECM. The complete decellularization of the animal tissues was verified both microscopically over H&E staining of the decellularized tissues that are devoid of visible cell nuclei and quantitatively in the DNA contents (<50 ng per mg of ECM) as described previously (17). The fluorescence intensity of DNA was measured at 430 nm after digestion of the extracted ECM with acidic pepsin solution (pH 6.5) at 65° C. for 24 hours. The quality of the ECM retrieval was further measured by analyzing the major ECM protein components, such as collagen and GAGs. The total ECM collagen was estimated using the conventional hydroxyproline assay (60). The GAGs content was analyzed as described (61) that quantifies the amount of sulphated glycosaminoglycans presented in tissues using 1,9-dimethylmethylene blue solution.

Mass Spectrometry.

i) Enzymatic “In Liquid” digestion of DBT-ECM and IrECM: The lyophilized DBT-ECM was solubilized in 8 M Urea containing 0.07% ProteaseMAX (Promega), 50 mM NH₄HCO₃ (pH 8.5) and 10 mM TrisHCl (pH 7.0) to the concentration of ˜10 mg/mi. The suspension was sonicated in a sonicator bath three times for 1 minute each. Meanwhile, 100 μg of IrECM extract (10 μl) was denatured in 30 μl of 8 M Urea and 4.4 μl of 1% ProteaseMAX. Both the DBT-ECM and the IrECM solutions were stored overnight at 4° C. to facilitate further solubilization. The homogenously reconstituted samples were taken for the downstream 400 μl digestion, where the samples were diluted to 240 μl for a reduction step with 10 μl of 25 mM DTT, 115 μl of 25 mM NH₄HCO₃ (pH 8.5), 20 μl of MeOH, 40 μl of 8 M Urea and 35 μl of 0.2% ProteaseMAX, incubated at 52° C. for 15 minutes, and cooled on ice down to RT. Then, 12 μl of 55 mM IAA was added for alkylation and incubated in dark at RT for 15 minutes. The reactions were quenched by adding 32 μl of 25 mM DTT. Subsequently, each sample was split into two portions. One portion was digested without further handling whereas the second portion was treated with 6 μl of PNGase F enzyme (Promega) at 37° C. for 2 hours. For protease digestion, 20 μl of Trypsin/LysC mix solution [50 ng/μl Trypsin from Promega and 50 ng/μl LysC from WAKO in 25 mM NH₄HCO₃] and 40 μl of 25 mM NH₄HCO₃ (pH 8.5) were added to a 200 μl of final volume. Digestion was conducted at 42° C. for 2 hours, followed by addition of 10 μl of trypsin/LysC solution, and further digested at 37° C. overnight. The reactions were terminated by acidification with 2.5% TFA [Trifluoroacetic Acid] (0.3% final concentration).

ii) NanoLC-MS/MS: The digested protein solutions (50 μg) were cleaned up using the OMIX C18 SPE cartridges (Agilent, Palo Alto, Calif.) per the manufacturer's protocol, eluted in 20 μl of 60/40/0.1% ACN/H₂O/TFA, completely dried in speed-vac, and reconstituted in 25 μl of 0.1% formic acid. Peptides were analyzed by NanoLC-MS/MS (Biotechnology Center, University of Wisconsin-Madison) using the Agilent 1100 Nanoflow system (Agilent) connected to a new generation hybrid linear ion trap-orbitrap mass spectrometer (LTQ-Orbitrap Elite™, Thermo Fisher Scientific) equipped with an EASY-Spray™ electrospray source. Chromatography of peptides prior to mass spectral analysis was accomplished using capillary emitter column (PepMap® C18, 3 μM, 100 Å, 150×0.075 mm, Thermo Fisher Scientific) onto which 2 μl of extracted peptides was automatically loaded. NanoHPLC system delivered solvents A: 0.1% (v/v) formic acid and B: 99.9% (v/v) acetonitrile with 0.1% (v/v) formic acid at 0.50 μL/min to load the peptides (over a period of 30 minutes), and 0.3 μl/min to elute peptides directly into the nano-electrospray with gradual gradient from 3% (v/v) B to 20% (v/v) B over 154 minutes, and concluded with 12 minutes fast gradient from 20% (v/v) B to 50% (v/v) B at which time a 5 minute flash-out from 50-95% (v/v) B took place. As peptides eluted from the HPLC-column/electrospray source, MS scans were acquired in the Orbitrap with a resolution of 120,000 followed by MS2 fragmentation of 20 most intense peptides detected in the MS1 scan from 380 to 1800 m/z; redundancy was limited by dynamic exclusion.

iii) Data analysis: Raw MS/MS data were converted to mgf file format using MSConvert (ProteoWizard: Open Source Software for Rapid Proteomics Tools Development). The resulting mgf files were used to search against Mus musculus amino acid sequence database with a decoy reverse entries and a list of common contaminants (87,154 total entries with 43,539 mouse proteins from UniProt database downloaded Sep. 18, 2014) using in-house Mascot search engine 2.2.07 (Matrix Science) with variable Methionine and Proline oxidation, and with Asparagine and Glutamine deamidation. Peptide mass tolerance was set at 15 ppm and fragment mass at 0.6 Da. Protein annotations, significance of identification and spectral based quantification was done with the help of Scaffold software (version 4.3.2, Proteome Software Inc., Portland, Oreg.). Protein identifications were accepted if they could be established at greater than 80.0% probability within 1% False Discovery Rate and contained at least 2 identified peptides. Protein probabilities were assigned by the Protein Prophet algorithm (Nesvizhskii, Al Anal Chem. 2003 Sep. 1; 75(17):4646-58). Proteins that contained similar peptides and could not be differentiated based on MS/MS analysis alone were grouped to satisfy the principles of parsimony. The spectrum counts presented in Table 1 and Table 2 were expressed as average values from the protein samples that were processed using deglycosylation and glycosylation methods.

TABLE 1

| | | | Spectrum |
|---|---|---|---|
| | # | Protein | Count |
| Collagen | 1 | Collagen type I alpha 1 chain | 899 |
| | 2 | Collagen type III alpha 1 chain | 812 |
| | 3 | Collagen type I alpha 2 chain | 769 |
| | 4 | Collagen type V alpha 2 chain | 143 |
| | 5 | Collagen type VI alpha 3 chain | 123 |
| | 6 | Collagen type II alpha 1 chain | 62 |
| | 7 | Collagen type V alpha 1 chain | 60 |
| | 8 | Collagen type VII alpha 1 chain | 56 |
| | 9 | Collagen type IV alpha 2 chain | 47 |
| | 10 | Collagen type V alpha 3 chain | 33 |
| | 11 | Collagen type IV alpha 1 chain | 27 |
| | 12 | Collagen type VI alpha 1 chain | 20 |
| | 13 | Collagen type VI alpha 2 chain | 15 |
| | 14 | Collagen type XI alpha 2 chain | 7 |
| | 15 | Collagen type XVI alpha 1 chain | 4 |
| | 16 | Collagen type XIV alpha 1 chain | 2 |
| | 17 | Collagen type XXII alpha 1 chain | 2 |
| | 18 | Collagen type XV alpha 1 chain | 1 |
| | 19 | Collagen type IV alpha 3 chain | 1 |
| | 20 | Collagen type IV alpha 5 chain | 1 |
| Glycoprotein & | 21 | Periostin | 66 |
| Proteoglycan/GAG | 22 | Laminin subunit gamma 1 | 26 |
| | 23 | Laminin subunit beta 1 | 13 |
| | 24 | Laminin subunit alpha 1 | 9 |
| | 25 | Laminin subunit beta 2 | 8 |
| | 26 | Laminin subunit alpha 5 | 5 |
| | 27 | Fibronectin | 23 |
| | 28 | Fibrillin 1 | 16 |
| | 29 | Fibrinogen alpha chain | 14 |
| | 30 | Fibrinogen gamma chain | 13 |
| | 31 | Fibrinogen beta chain | 9 |
| | 32 | Nidogen 1/Entactin | 9 |
| | 33 | Tenascin-X | 2 |
| | 34 | EMILIN 1 | 1 |
| | 35 | Perlecan | 30 |
| | 36 | Lumican | 13 |
| Other ECM | 37 | Titin | 9 |
| Proteins | 38 | Perilipin 1 | 5 |
| | 39 | Perilipin 4 | 3 |
| | 40 | Elastin | 3 |
| | 41 | Dermatopontin | 3 |

The major proteins identified in mouse mammary tissue ECM that are preserved in TMS. The proteins were grouped according to their similarities in a family or functions in the ECM, and listed from high to low spectrum counts.

TABLE 2

| | | | Spectrum |
|---|---|---|---|
| | # | Protein | Count |
| Collagen | 1 | Collagen type IV alpha 2 chain | 87 |
| | 2 | Collagen type IV alpha 1 chain | 55 |
| | 3 | Collagen type I alpha 1 chain | 35 |
| | 4 | Collagen type I alpha 2 chain | 15 |
| | 5 | Collagen type III alpha 1 chain | 9 |
| | 6 | Collagen type XVIII alpha 1 chain | 7 |
| | 7 | Collagen type VI alpha 1 chain | 5 |
| | 8 | Collagen type II alpha 1 chain | 2 |
| | 9 | Collagen type XII alpha 2 chain | 2 |
| | 10 | Collagen type XV alpha 1 chain | 2 |
| Glycoprotein & | 11 | Laminin subunit alpha 1 | 1567 |
| Proteoglycan/GAG | 12 | Laminin subunit gamma 1 | 1324 |
| | 13 | Laminin subunit beta 1 | 1205 |
| | 14 | Laminin subunit alpha 5 | 28 |
| | 15 | Laminin subunit beta 2 | 10 |
| | 16 | Laminin subunit alpha 4 | 3 |
| | 17 | Nidogen-1 | 342 |
| | 18 | Nidogen-2 | 47 |
| | 19 | Perlecan | 284 |
| | 20 | Fibronectin | 84 |
| | 21 | Fibrinogen beta chain | 40 |
| | 22 | Fibrinogen gamma chain | 33 |
| | 23 | Fibrinogen alpha chain | 15 |
| Other | 24 | Alpha-1-antitrypsin 1-4 | 16 |
| ECM | 25 | Vitronectin | 6 |
| Proteins | 26 | Fibulin-1 | 5 |

Major proteins identified in Matrigel. Proteins are grouped according to similarities in family or function in the ECM, and listed from high to low spectrum counts.

Characterization of the porosity and compliance of the TMS. The DBT-ECM powder was homogenized in acidic pepsin solution at the final concentration of 50, 100 and 150 mg/ml, respectively, followed by neutralization, moulding, freeze-drying, and cross-linking (UV irradiation for solid porous and tyrosinase for hydrogel forms of TMS, respectively) as described above. The scaffolds were either subjected to scanning electron microcopy (SEM) or optimal cutting temperature (OCT) compound embedded, cross sectioned, H&E stained, and imaged under light microscope. For SEM, the scaffolds were fixed with 2.5% glutaraldehyde for 30 minutes, washed 5 times in distilled water, kept at −80° C. for an hour, and freeze-dried for at least 24 hours. The dried scaffolds were then exposed to SEM under low vacuum to take the images. The compliance or sponginess of the TMS scaffolds was demonstrated by retaining the shapes after gently pressing and releasing the scaffolds with the forceps (Video S1).

Scaffold fabrication from cell cultures. The cell ECM-based scaffolds were generated following the tissue TMS fabrication protocol with modifications on the decellularization process. The MM231 cells were collected by scraping from tissue culture dishes, pelleted by centrifugation, and subjected to rapid freeze and thaw cycles (RFTC, 5-10 minutes in liquid nitrogen, and 20-30 minutes on ice depending on the cell pellet size). After three rounds of RFTC, the pellet was washed three times in 1×PBS containing 0.05% of SDS, followed by another three rounds of the RFTC and washing in 1×PBS. The decellularized MM231 ECM was pelleted, characterized, and processed for scaffold generation as for the decellularized tissue ECM.

Fabrication of 3D Scaffolds using collagen or IrECM hydrogel. Collagen powder was dissolved in the acidic pepsin solution as described above. Freeze-dried IrECM was reconstituted in distilled H₂O. Both the collagen and the IrECM hydrogel solutions are prepared at the same concentration as that of the hydrogel derived from the decellularized breast tissues or the MM231 cell cultures for the production of porous scaffolds as described above.

Fabrication of 3D Scaffolds from synthetic polymers. PCL and PLGA were dissolved in chloroform at the final concentration of 0.5 g/ml and 1 g/ml, respectively. Sodium bicarbonate (1 g/ml) was then added into the PCL and PLGA solution, and mixed. The solutions were dispensed slowly into the Sigmacote-coated moulds as described above, and freeze-dried at −50° C. for 48 hours to obtain spherical scaffolds. The scaffolds were washed in 0.1 N hydrochloric acid solution at RT for 6 hours (replacing the solution hourly), followed by washing in distilled water for several times until the pH of the water became neutral. The scaffolds were soaked in 70% ethanol for 3-5 hours, washed with 1×PBS for 3-5 times, and kept in 1×PBS until use.

In Vitro 3D Tissue Culture.

i) Regular 3D culture. The porous DBT-TMS, DMT-TMS, DMM231, collagen, IrECM, PLGA, and PCL scaffolds were washed with sterile 1×PBS for several times, pre-conditioned with culture medium in 24-well culture plates, a process that allows the settling down of the scaffolds at the bottom of the plates. 1×10⁵ cells (human GM637 fibroblasts, mouse NIH/3T3 fibroblasts, MCF10A, or MM231 cells) in 10-20 μl of medium per scaffold (cell number can be adjusted according to the size of the scaffold used) were seeded onto the scaffolds after removing the medium from the wells. The cell-laden scaffolds were placed in tissue culture incubator (37° C., 5% CO₂) for 45 minutes to allow the cells to attach to the scaffolds. Then optimal culture medium was added, and replaced according to the experimental plans. The cultured samples were collected at indicated time points, analyzed, or used in downstream experiments.

ii) Compartmental 3D tissue culture. This is a multilayered scaffolding design involving the use of both porous and hydrogel forms of TMS derived from the same tissue ECM. After the cells were seeded on the porous scaffold as described above, a layer of hydrogel (0.5-1 mm thick) with or without cells was applied outside the cell-laden porous TMS, followed by coating with additional layer(s) of the hydrogel with or without cells as desired. In this study, MM231 cells were seeded on the porous TMS, and human or mouse fibroblasts were blended into the outer layer of the hydrogel, spaced with a blank hydrogel layer for the sake of observing cell proliferation, migration and invasion into the blank gel layer. To ensure the spongy structure of the hydrogel layer(s) and good conductivity of nutrients and O₂, tyrosinase (2 parts, final concentration 50 U/ml) was added into the mixture of cold culture medium (2 parts) and the DBT-TMS hydrogel (6 parts) on ice, and kept in dark before coating onto the porous scaffold. The hydrogel coating was conducted in the hemispherical moulds of the porcelain file with bigger diameter than the scaffolds to be coated. For instance, the 2 mm diameter cell-laden scaffolds can be coated in 3 or 4 mm diameter moulds. If a centralized positioning of the cell-laden TMS is desired, a two-step hydrogel coating can be applied, where the bottom and the top portion of the hydrogel were casted separately. The file carrying the TMS assemblies was placed into incubator (37° C., 5% CO₂) for 45 minutes, during which the tyrosinase cross-linked the hydrogel. Then, the assemblies were transferred into the wells of culture plates, and cultured under optimal conditions as described above.

Proliferation assay in 3D culture. The proliferation of the cells grown on the different scaffolds or treated with the different drugs was measured using the CCK-8 reagent at the time points indicated. Briefly, the cell-laden scaffolds (triplicates for each condition) were cultured in 96-well plates. CCK-8 solution was added at a 1:10 dilution into the cultures. The colorimetric absorbance of the supernatants of the cultures that reflects the cell proliferation rate was measured at 490 nm using a Synergy 2 microplate reader (BioTek) after 4 hours of incubation (37° C., 5% CO₂). Error bars represent standard deviations (s.d.) of the means of three independent experiments.

Live/Dead Cell staining. The survival of the cells grown on the 3D scaffolds were assessed using the Live/Dead Cell Staining kit. The cell-carrying scaffolds at different time points or under different treatment conditions were collected and manually sectioned at 250-500 μm of thickness. The sections were washed twice with 1×PBS (37° C.), incubated in the staining solution (2 μM of calcein-AM and 4 μM of EhtD-III in 1×PBS) at RT for 45 minutes, and imaged using fluorescence microscopy. Calcein-AM stains live cells green (under EGFP filter), while EthD-III stains dead cells red (under Texas Red filter).

In vivo experiments. 1×10⁵ MM231 cells/scaffold were seeded on 2 mm-diameter spherical porous scaffolds (DBT, DMM231, and PLGA), and cultured under optimal conditions for 24 hours prior to implantation. Another group of the cell-laden scaffolds were coated with a layer of DBT-hydrogel an hour before the implantation as described before. The blank scaffolds without cells were used as negative controls. The scaffolds were implanted into the mammary fat pads of the 8 weeks old female NOD-SCID mice (Charles River Laboratories). Each implantation condition was tested in triplicates over 3 different mice. The implants and tumors were assessed (size measurement with caliper and CT imaging), retrieved 4 weeks post-implantation, and subjected to histological processing and extended analysis.

Histology and immunostaining. The cell-laden scaffolds from the tissue cultures and the harvested tumors from mice were washed twice with ice-cold 1×PBS, and fixed in 10% neutral buffer formalin (NBF) solution for 24-48 hours at 4° C. After rinsing with cold 1×PBS, the 3D cultures and the tumor samples were embedded into OCT or paraffin following standard protocols, and sectioned at 10 μm of thickness using cryostat or microtome. For the sections produced using the paraffin fixation, a deparaffinization and rehydration process was performed, followed by antigen retrieval using the Tris/EDTA buffer (10 mM Tris Base, 1 mM EDTA solution, and 0.05% Tween 20; pH 9.0). The sections were washed several times with water, stained with H&E or immunofluorescence antibodies (corresponding primary and Alexa fluorophore-conjugated secondary antibodies) as described previously (7), and imaged using light or fluorescence microscopy for further analysis. The capillary and solid tissue occupancies (surface areas) on 3 consecutive cross sections (H&E stained) of 3 replicate tumors were quantified using the ImageJ 1.49v software (National Institutes of Health, USA) for statistical significance.

Drug screening. The T47D and BT474 breast cancer cells were used to test the efficacies of the two anticancer drugs, HT and Taxol, in 2D and 3D cultures. 2×10³ cells/well and 1×10⁵ cells/scaffold (in triplicates) were seeded on 2D surface and the 3D scaffolds, respectively, and cultured in 96-well plates for 7 days. The drugs at the final concentration of 1 μM were administered separately to the cultures on day 7, 9, 11, 13 of culture for 24 hours, and cell survival as well as proliferation status assessed on day 1, 8, 10, 12, 14, 21 using the CCK-8 reagent and the Live/Dead assay, respectively. After the measurement on day 14, a seven-day recovering period was included to evaluate the post-treatment proliferation potential of the cells. Three independent experiments were performed for statistical significance.

Statistical analysis. The statistical data were expressed as mean±s.d. with one-way analysis of variance (One-way ANOVA) using the StetPlus, AnalystSoft Inc. (Build 6.0.0/Core v5.9.92, Walnut, Calif.). Error bars represent the s.d. of the means.

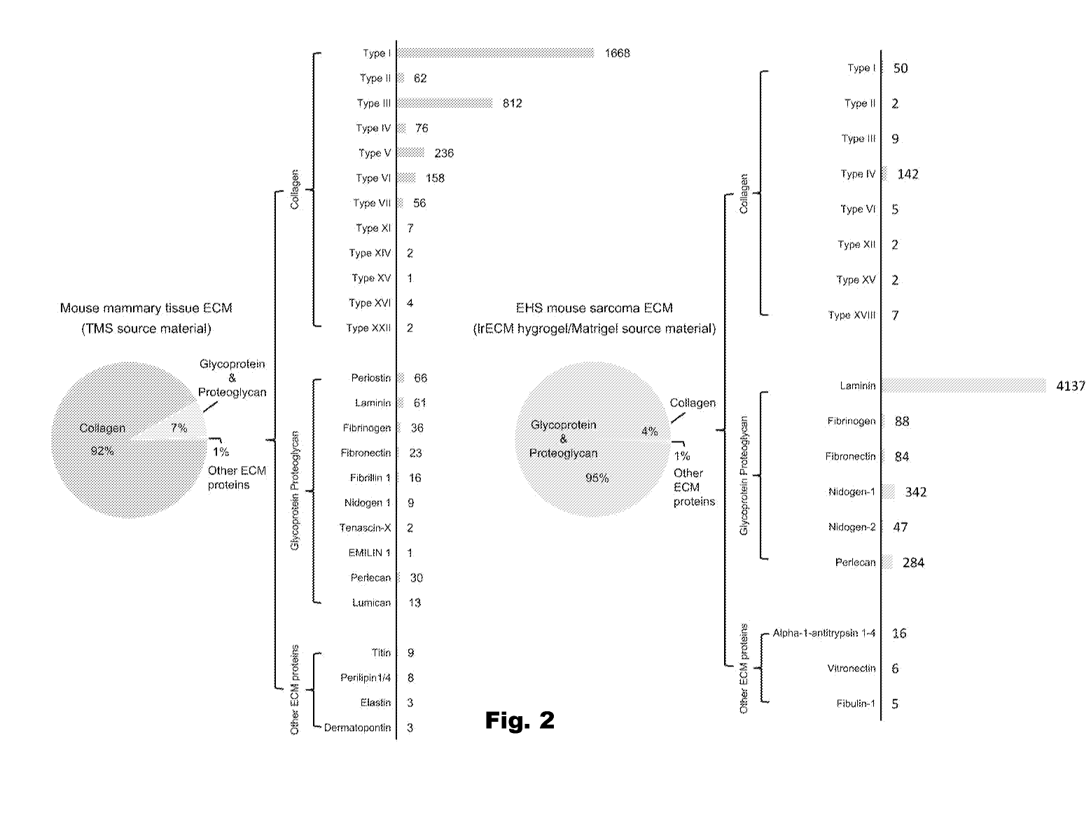

The decellularization and removal of DNA contents from the tissues are critical steps during tissue ECM extraction. Mice breast tissues were collected and decellularized (FIG. 1a) as described in the methods (see Materials and Methods). Compared to the native tissues, an absence of the visible nuclei in the decellularized tissues as checked using hematoxylin and Eosin (H&E) staining (FIG. 1d, bottom panels) and the total DNA content of <50 ng per mg of dried ECM (>99% of DNA was removed, FIG. 1b) satisfy the decellularization criteria (24). The amount of the main ECM components, such as collagen and glycosaminoglycans (GAGs), retained in the decellularized matrix is very close to that of the native tissues as measured with the conventional hydroxyproline assay and the 1,9-dimethylmethylene blue method, respectively (FIG. 1b). Histological examination of cross sections of decellularized mouse muscle and mammary tissues showed the absence of cells within tissues, while their ECM architectures remain well preserved (FIGS. 1c, 1d). To better characterize the extracted ECM composition, liquid chromatography-mass spectrometry (LC-MS/MS) was carried out. The ECM proteins within the mouse breast tissues were identified (Table 1), which were abundant in different types of collagens, with certain amounts of glycoproteins (including periostin, laminin, fibronectin, fibrillin, and fibrinogen, etc) and proteoglycans/GAGs (such as perlecan and lumican) as well as other less abundant yet important proteins for the structures and functions of the ECM. In contrast, the IrECM is rich in the basement membrane proteins, such as laminin, perlecan, nidogen, and type IV collagen, poor in overall collagen content and amount, and has other ECM proteins different from those in the breast tissue ECM (Table 2 and FIG. 2). Clearly, the breast tissue ECM proteins are more diverse and cover broader components of the connective tissues than those in the IrECM hydrogel, highlighting the distinct biochemical features and potential differences in the structural supports of the two ECM-based systems. It was also observed that while both types of the ECMs contained certain cell cytoskeleton proteins, a range of intracellular proteins was identified in the IrECM hydrogel (data not shown). The physical localizations of these proteins, among which a small number could be cell-secreted, deserve to be further explored. These findings indicate that understanding the composition of a select ECM is important for addressing tissue-specific and disease-relevant questions since many cellular activities conducted for the survival and growth of the cells living within a tissue environment are initiated by the biochemical changes besides the ECM structural modifications in the extracellular space. Next, TMS was generated in two forms, hydrogel and porous scaffolds, with the decellularized breast tissue ECM (FIG. 1a. The porous TMS can be fabricated in different shapes and sizes with desired porosity, which can be achieved by adjusting the amount of the ECM dissolved in the acidic solution as analyzed by scanning electron microscopy (SEM, FIG. 1c). The sizes of the TMS pores were inversely proportional to the ECM amount in the scaffolds, with 100 mg of ECM per ml of the solution gave rise to scaffolds with pore sizes of about 100 μm (FIG. 1c) that were used across the different experiments in this study. Both the porous and the hydrogel scaffolds became spongy in water, PBS solution, and tissue culture medium, and were stable for months without any noticeable deformation and degradation. The cross-linking and architectural stability of the ECM can be further enhanced by adding tyrosinase during the fabrication process. Tyrosinase is a physiological oxidase found in melanocytes for melanin generation, and has been used as a protein cross-linker to preserve molecular features of proteins (25). Tyrosinase makes the porous TMS spongy with a stable architecture, becomes inactive within hours at 37° C., is diluted through changing media, and had no detectable effects on cell morphology, growth, and proliferation under the concentration and culture conditions used in the study (data not shown).

H&E staining of the cross sections of the porous TMS reconstituted from the decellularized breast tissue ECM (DBT-TMS) revealed close mimicry of the structural characteristics of the decellularized native tissues (FIG. 1d, left panels). Human GM637 fibroblasts (or NIH/3T3 mouse fibroblasts, not shown) seeded on the surface of the porous TMS attached and grew very well, and infiltrated into the scaffold in days as demonstrated by the H&E staining of the cross sections of the cell-laden TMS (FIG. 1d, top-right panel), which highly resembled that of the breast tissues (FIG. 1d, bottom-right panel). Close-up inspections of the fibroblasts that infiltrated into the TMS (FIG. 1e, top-left panel) revealed similar cell distribution as that of the stromal cells in normal breast tissues (FIG. e, bottom-left panel). SEM analysis of breast cancer MDA-MB-231 (MM231) cell-laden TMS showed both surface and intra-porous occupancies of the cells (FIG. 1e, top-right panel). This phenotype was consistent with the scattering pattern of the cancer cells in breast tumors originated from the MM231-laden TMS implants in mouse mammary fat pad, where the cancer cells were distinguishable from the surrounding stromal cells by Ki-67 (green, positive for the MM231 and negative for normal stromal cells) and HER2 (red, positive for the stromal cells and negative for the MM231 cells) immunofluorescence (IF) staining (FIG. 1e, bottom-right panel). These data collectively indicate that the tissue-like architecture and the compliance of TMS ensure its mechanistic mimicry of the tissue's physical environment that is essential for force-mediated signaling regulation of cell attachment, survival, and migration in the 3D space (26, 27). The essential physicochemical features of a culture substratum are critical for the expression of distinct biomarkers that are otherwise not induced or hard to be detected in 2D or non-biologically relevant 3D cultures.

### Example 2: Proliferation and Growth Status of Cells in TMS

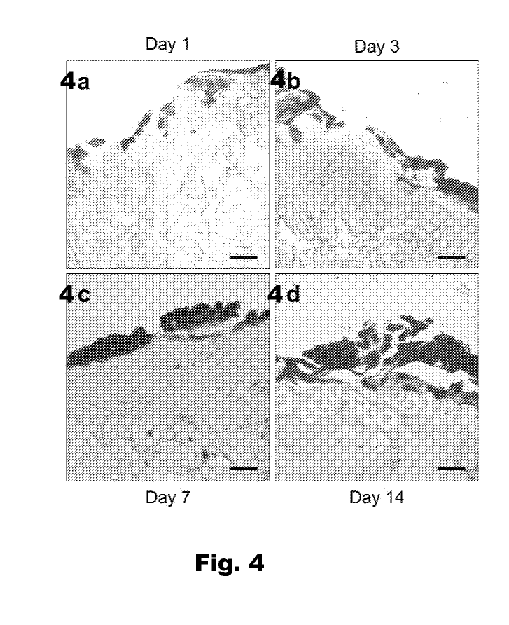

The normal mammary epithelial MCF10A cells and the MM231 cancer cells were seeded on separate TMSs (FIG. 3a), and evaluated for proliferation using the Cell Counting Kit-8 (CCK-8) over 2 weeks of culture in optimal conditions. The results showed that both types of the cells proliferated substantially in the TMS cultures, with the MM231 cells proliferated at higher levels (FIG. 3b), possibly due to the aggressive growth nature of the cells. H&E staining of the cross sections of the samples also exhibited a marked increase in cell numbers within the TMSs in 2 weeks (FIGS. 3c-3f). Of note, the scaffolding TMS structure diminished over the culturing time frame, indicative of the gradual degradation of the exogenous ECM and the establishment of novel ECM by cancer cells. In contrast, the cells grown on the PLGA scaffolds had fairly packed accumulation at the surface of the scaffold with limited distribution inside the scaffolds during the time of the observations (FIGS. 4a-4d). The survival status of the MM231 cells grown on the TMSs was investigated using Live/Dead Cell staining, where the green and red fluorescent probes labeled the live and dead cells, respectively, followed by fluorescence microscopy. The cancer cells gradually occupied the surface and inner space of the TMSs over time (FIGS. 3g-3j). Interestingly, only few dead cells were detected during the course of the observations (FIGS. 3i, 3j), indicating robust cell survival and propagation were established in the cultures. The proliferation of the MCF10A and the MM231 cells grown on the TMSs (decellularized mouse breast tissue, DBT; decellularized muscle tissue, DMT) was compared with those on other 3D porous scaffolds generated from the natural ECM component (collagen or IrECM), decellularized MM231 ECM scaffolds (DMM231), and the synthetic polymer scaffolds (PLGA or/and PCL). At the indicated time points, cell proliferation on the scaffolds was measured using CCK-8. The results showed that there was an increase in cell numbers across all the types of the scaffolds tested over time (FIGS. 3k-3n). The MM231 cells grown on the DMM231 scaffolds had the greatest cell proliferation rate compared to those on the other scaffolds (FIGS. 3k-3n). A similar phenotype was reported in the MCF7 breast cancer cells cultured on decellularized tumor tissues (28). These observations indicate that cancer cells grow better within a tumor supporting tissue microenvironment. The DBT-TMS also supported cancer cell proliferation to a higher extent than the collagen or the IrECM scaffold-based cultures, especially after a week of culturing period, while the DMT-TMS cultures displayed moderate increase in cell numbers (FIGS. 3k-3n). All of the scaffolds derived from the natural biomaterials showed better support for the proliferation of both the MCF10A and the MM231 cells than the synthetic polymer-based scaffolds. The MCF10A cells displayed similar proliferation patterns among the collagen, IrECM, DMT and DBT group as well as the PCL, PLGA and PCL+PLGA group of scaffolds. Still, the DBT scaffolds demonstrated the best support for the proliferation of the MCF10A cells across the different cultures (FIGS. 3k-3n). These data collectively indicate that the full tissue matrix-based TMS is a competent 3D culture system supporting robust cell survival and proliferation in a tissue-mimicking microenvironment.

### Example 3: Compartmental Co-Culture of Cancer Cells with Stromal Cells in Multilayered TMS

In addition to interacting with the ECM and the cells of the same origin, cancer cells living in a tissue also interact with the stromal (e.g. fibroblasts) or other types of cells, a process that is essential for inter- and intracellular signaling as well as for the growth, proliferation, migration and invasion of the cancer cells. It was shown that tumor-derived stroma was able to induce desmoplastic differentiation and morphological changes of normal fibroblasts, and displayed matrix characteristics supporting migratory and proliferative phenotypes of cancer cells that were reminiscent of tumor progression (29). On the other hand, carcinoma-associated, but not normal, fibroblasts stimulated tumorigenesis from initiated epithelial cells (30), implicating a tumor stroma-directed transformation and promotion of cancer development. Additionally, the interactions of fibroblasts, macrophages, or/and endothelial cells with breast cancer cells in co-cultures promoted the secretion of tumor-promoting factors from the cancer cells as well as their proliferation and migration (31). These observations indicate that including stromal cells in a culture system when studying tumor biology can be useful to reveal the signaling-oriented molecular mechanisms governing tumor progression.

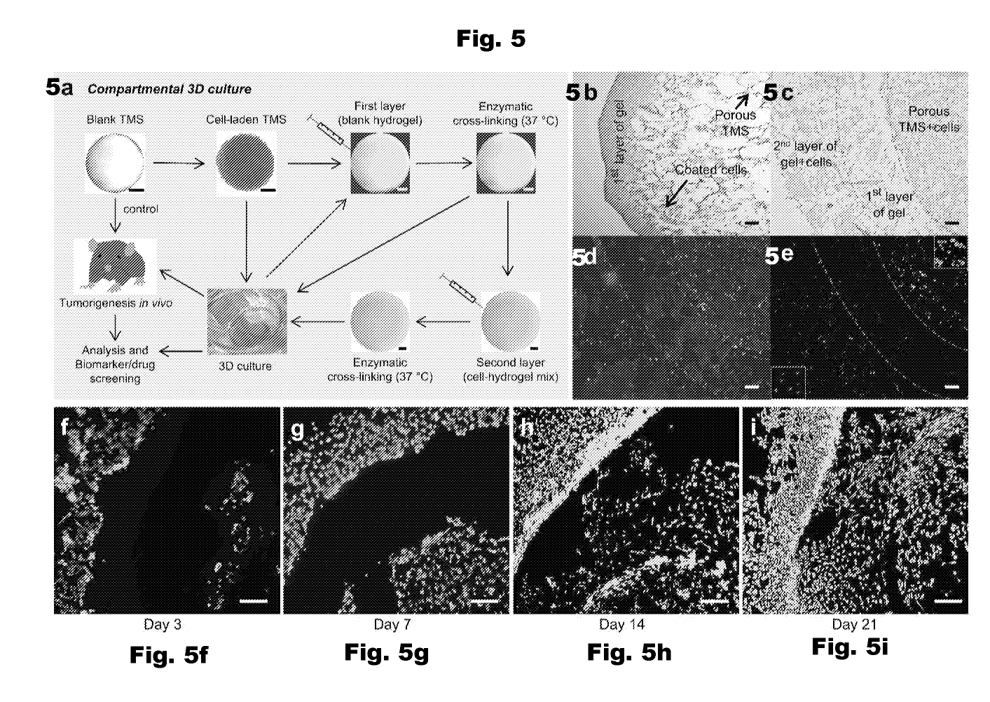

To mimic the complex cell-cell/ECM interaction in tissues, different co-culture models have been developed, including the collagen/hyaluronic acid (HA) scaffold (32), Matrigel method (33), heterogeneous spheroid (34), nanoshuttle-magnetic levitation model (35), etc. While these models support cell growth and interactions in 3D, the straight mix of cell populations in a single compartment limits the dynamic and detailed observations of cell-cell and cell-matrix interactions. The microfluidic flow cell method allows compartmentalized culture of cells (36, 37), but does not support direct interactions and free migration of different cells that could potentially affect the ECM disposition and the biological behaviors of the cells. Besides, the model requires special apparatus, and may cause shear stress to the cells when injecting the cell-gel mixture into the flow wells. Recently, the generation and application of layered hydrogel scaffolds have been described by Ladet et al (Chitosan gel)(38), Fang et al (alginate gel)(39), and Todhunter et al (DNA-programmed assembly of cells in Matrigel-collagen mixture)(40), respectively. While these layered culture models are able to provide physical support for cells to grow, their ECM compositional cues are insufficient. The models therefore lack the essential signaling supports for tumor initiation and the establishment of cell-specific ECM for the growth of tumoroids in culture. Taking the advantage of fabrication methods disclosed herein that can generate TMS in both porous and hydrogel forms from the same tissue ECM (see Materials and Methods), a multilayered tissue culture platform has been produced. As illustrated in FIG. 5a, 1×105/scaffold of MM231 cells were seeded on the porous DBT-TMS, followed by either straight 3D culture or coating one or more layers of TMS hydrogel (FIGS. 5a-5c). Normally, the medium-diluted hydrogel does not gelatinize well because of the poor cross-linking efficiency of the ECM proteins. By adding tyrosinase into the TMS hydrogel and culture medium mixture, the hydrogel was polymerized and displayed tissue-like resilience. The first gel layer without cells served as an inter-compartmental region for the observations of cell migration and invasion (FIG. 7b). A second layer of hydrogel containing a different type(s) of cells (e.g. human GM637 or mouse NIH/3T3 fibroblasts) was applied (FIGS. 5a, 5c). After gel polymerization, the multilayered assembly was cultured in optimal medium, which was replaced 12 hours after the initial culture. The distribution of the cells within the multilayered TMS after 3 days of culture was checked by DAPI staining of the cell nuclei (FIG. 5d). The progressive cell growth, proliferation, migration, and invasion into the first layer of the hydrogel were inspected using Live/Dead cell staining of the cross sections of the scaffolds (FIGS. 5f-5i). The robust cell survival, proliferation and mobility (spindle-shaped morphology of the migrating cells) within the TMS indicate the easy accessibility to nutrients and oxygen by the cells across the different compartments of the assembly. Alternatively, the cell-laden TMS with or without hydrogel-coating can be used for biomarker/drug screening, or implanted into animals for tumor formation and therapeutic testing (FIG. 5a). One of the important profiles of the biological activities conducted by a specific cell population is the expression of select biomarkers. To assess cellular biomarker expression within the TMS, MM231 cells and GM637 cells were seeded on the porous TMS and in the second layer of the hydrogel, respectively, spaced with a blank hydrogel layer. After 3 days of culture, the TMS was harvested, cross sectioned, and immunofluorescently stained for the cell proliferation marker Ki-67 and the HER2 receptors. Consistent with clinical evidence (41), the MM231 cells were stained positive for Ki-67 (FIG. 5e, green) and negative for HER2 (red), and the GM637 cells were positive for HER2 and negative for Ki-67. It was also noticeable that both the MM231 and the GM637 cells started migrating into the blank gel layer of the TMS (FIG. 5e). Together, these data demonstrate that the compartmental TMS system not only can mimic the “layered tissue” structures (e.g. the epithelial cell, basement membrane and connective tissue unities in certain parts of human or animal tissues, such as the mammary tissues), but also is a convenient tool for the observations of multiple phenotypes of different cell populations in a single system and for the screening of tumor biomarkers.

### Example 4: TMS Support of Tumor Formation in Animals

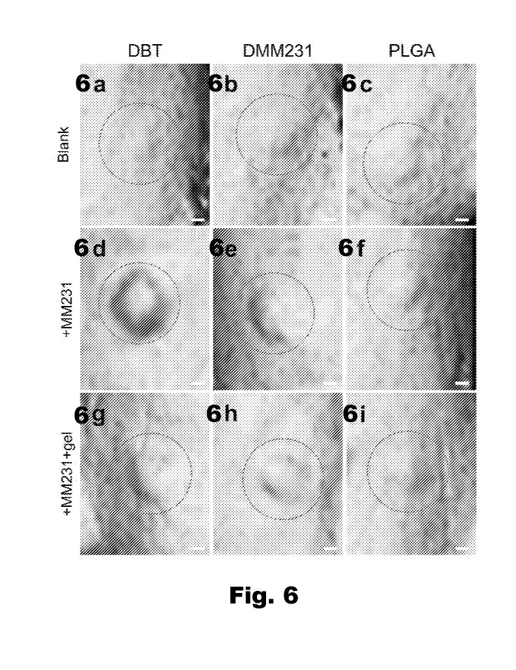

Injection of human breast cancer cells into mice mammary fat pad to induce tumor formation has been commonly used in the field (42). Yet, this method may cause shear and survival stresses to the cells precultured in 2D substratum, and the tumor induction takes long time (usually above 6 weeks before collection) with quite variable sizes. Similarly, Matrigel and collagen plugs carrying breast cancer cells form tumors in animals over extended period of time (43). Synthetic polymer scaffolds were also tested in supporting tumor development in animals. However, the large number of cells used, the longer than 2 weeks of preculturing of the cells on the scaffolds and limited tumor mass formation in weeks indicate that the scaffolds may not provide optimal growth conditions to the cells (44). As the TMS and the DMM231 scaffolds demonstrated superior support for cell survival and proliferation in tissue cultures, the efficiencies of the scaffolds in supporting tumor development in mice were further tested. MM231 cells were seeded on the porous DBT-TMS, DMM231 and PLGA scaffolds, respectively, with or without coating of a layer of DBT hydrogel as illustrated in FIG. 5a. The blank scaffolds (negative control) and the scaffolds containing the cancer cells in replicates were cultured in optimal conditions for 24 hours, and implanted into the mammary fat pads of the female mice. A group of MM231 cell-laden scaffolds with a hydrogel layer covered outside the seeded cells was also included in the experiments to test whether it can be integrated/degraded into the animal tissue well, penetrated by local nutrients for tumor growth, and potentially reduce host tissue response to xenograft tumors. Tumor formation within the tissues was analyzed at multiple levels after 4 weeks of implantation. First, the status and sizes of the tumors of the individual animal groups were analyzed with x-ray-based computed tomography (CT, FIGS. 6a-6i) prior to surgical excision, caliper measurement, and observation under dissection microscope. As shown, the blank DBT-TMS and DMM231 scaffolds were close to complete degradation and absorption, whereas the blank PLGA-scaffolds were partially degraded (FIG. 7a, upper panels), indicating that the tissue ECM-derived scaffolds have excellent biodegradability and biocompatibility within the host tissues. The tumors developed from the MM231 cells seeded on the DBT-TMSs were significantly bigger than those supported by the MM231-TMS and the PLGA-scaffolds, where the latter corresponded with the least tumor size (FIG. 7a, middle panels and FIG. 7b). A similar trend was found in the tumor groups derived from the MM231 cells plus a hydrogel layer outside the individual type of the scaffolds (FIG. 7a, lower panels and FIG. 7b). By and large, addition of the TMS hydrogel layer seemed to slow down tumor growth, which was almost deficient in the PLGA-scaffold group (FIG. 7a, upper panels; FIG. 7b). It is interesting that the DMM231 scaffolds supported better cell proliferation than the DBT-TMSs in tissue cultures (FIGS. 3k-3n), while the tumors originated from the DBT-TMSs were bigger than those from the DMM231 scaffolds (FIGS. 7a, 7b). These differences could be due to two major reasons. One is the nature of the scaffolds that allows the cancer cells grow better on the supporting ECM derived from their own living environment. MM231 cells grown on the DBT-TMS, though have richer ECM protein supports than the cells on the other types of scaffolds tested, may need to adapt to the tissue ECM condition to establish a fit cancer cell ECM environment. Once an optimal growth microenvironment is formed, the cells will gain exponential propagation as reflected on the 7th and 14th days of the 3D cultures shown in FIGS. 3c-3f. Another reason for the in vitro and in vivo differences may lie in the tissue environment that is in favor of the DBT-TMS support for cell growth. After the cancer cell-laden TMS was embedded into the mammary tissue, it is possible that the existing tissue ECM within the DBT-TMS permitted rapid infiltration of the surrounding fibroblasts, endothelial cells and other cells necessary to support the expansion of the cancer cells with minimum efforts to build up the ECM networks since most of the ECM components were already locally available. Under this situation, the cancer cells on the surface of the TMS can migrate more efficiently both inward into the scaffold and outward into the surrounding host tissues (FIGS. 8a, 8b), where they could potentially stimulate the fibroblasts to proliferate and to differentiate into cancer associated fibroblasts (45), which then generate collagen for the migration and spread of the cancer cells (46). The DMM231 scaffold in the animals, on the other hand, though can support the growth of the MM231 cells well, may not be an ideal environment for the infiltrated stromal cells, which will need to produce the required stromal ECM proteins to satisfy the expansion of the tumor mass.

Another intriguing finding was that the addition of the TMS hydrogel layer seemed to slow down the tumor growth, which was almost deficient in the PLGA-scaffold group (FIG. 7a, lower panels and FIG. 7b). This phenotype may in part be due to the physical constraint of the gel on the growth of the tumors, and to the possibly retarded accessibilities of the cells to the surrounding nutrients and O2 supply.

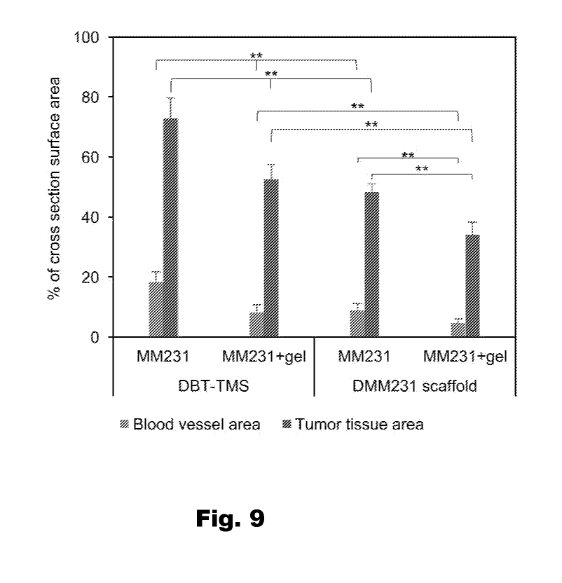

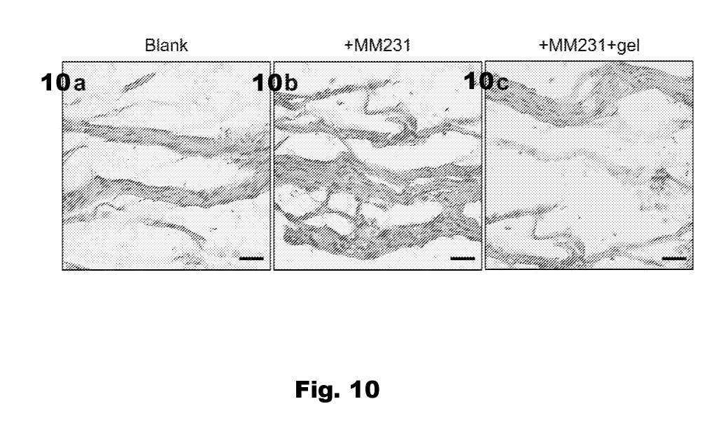

Second, histological examination of the cross sections of the tumor tissues revealed that the tumors of the DBT-TMS+MM231±gel groups had thicker and more organized ECM structures as well as richer capillaries close to the outer regions of the tumors compared to those of the DMM231 scaffold+MM231±gel groups (FIG. 7c ci-iv, FIGS. 8a-8d and FIG. 9). The matrix structures of the DBT-TMS and the DMM231 scaffolds were already hard to be discerned from the native tumor tissue ECM (FIG. 7c i-iv, FIGS. 8a-8d). These striking tumor tissue characteristics are clearly distinguishable from the normal breast tissue structures (FIG. 1d, bottom-right panel), where rich adipose and less connective tissues are present (47). The slightly loose intratumoral structures within the DMM231 scaffold-supported tumors could be a reflection of the under-developed tumor ECM (FIGS. 7c ii, 7c iv) as speculated above. Noticeably, there is a cell-dense zone close to the outer sections of the tumors, with fewer cells distributed along the inward radius toward the center of the tumor (FIGS. 8a-8d). Within the tumors developed from the MM231 cell-laden scaffolds covered by hydrogel, the cell population close to the outer edge of the tumors seemed to be rallied expanding outward (FIGS. 8c, 8d). The ECM architecture within the tumors of the PLGA-scaffold groups was not well maintained with barren capillaries that generated challenges for tissue sectioning (FIGS. 10a-10c). It appeared that the capillaries within the tumors developed more efficiently in the absence of the hydrogel coverage as clearly contrasted between the DMM231 scaffold+MM231±gel tumor samples (FIG. 7c ii vs. iv and FIG. 9), and the degree of the tumor vascularization was proportional to the tumor size (FIGS. 7c, 7a). Additionally, a better tumor ECM architecture was linked to enhanced microvessel formation (FIG. 7c and FIG. 9). Third, IF staining of Ki-67 (green) and HER2 (red) on the cross sections of the DBT-TMS/DMM231 scaffold+MM231 tumors demonstrated that the cancer cells are interwoven with fibroblasts, endothelial cells and other cells, and displayed identifiable capillary structures (FIG. 7c v, vi and FIGS. 8e, 8f). These results are in agreement with the observations that the infiltrated fibroblasts and macrophages was able to induce angiogenesis (48, 49), and promote cancer cell proliferation through secretion of growth and other tumor promoting factors (46, 50). Interestingly, some of the fibroblasts and endothelial cells within the tumors of the DMM231 scaffold+MM231 type were co-stained (orange color) for both Ki-67 and HER2, implicating that the cells were highly proliferative so as to meet the needs for stroma and microvessel generations (FIG. 7c vi). In contrast, within the tumors derived from the DBT-TMS/DMM231 scaffold+MM231+gel implants, most of the Ki-67 positive cancer cells accumulated toward the edge of the tumors (consistent with the phenotypes observed in FIGS. 8c-8d), while some remained mixed with stromal cells in deeper regions (FIG. 7c vii, viii and FIGS. 8g, 8h) where some fibroblasts and endothelial cells were co-stained for Ki-67 and HER2 (FIGS. 8g, 8h). These data are consistent with previous reports, which indicate that the cancer cell-oriented stroma could induce differentiation of normal fibroblasts (29), stimulate tumorigenesis of the initiated epithelial cells (30), and was dynamically modified with improved microenvironment (51) essential for the tumor promoting gene expression, cell migration, invasion (52, 53), and tumor growth (54). The necessity of a favorable stroma niche for the establishment of a tumor is also supported by the observation that the colonization of the metastatic cancer stem cells in a secondary site required secretion of select signaling enzymes into the ECM by the local fibroblasts (55). Together, these results indicate that the full tissue ECM-based scaffolds are biocompatible and biodegradable at a better level than the PLGA scaffolds, and support better interactions between the tumor cells and stromal or other types cells that all contribute to more robust tumor growth.

### Example 5: Drug Screening Study

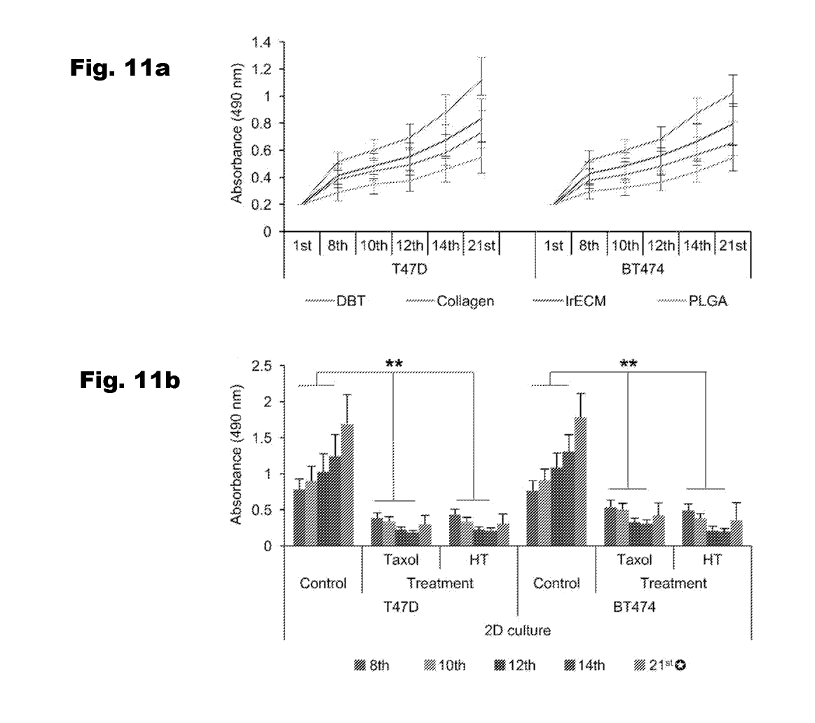

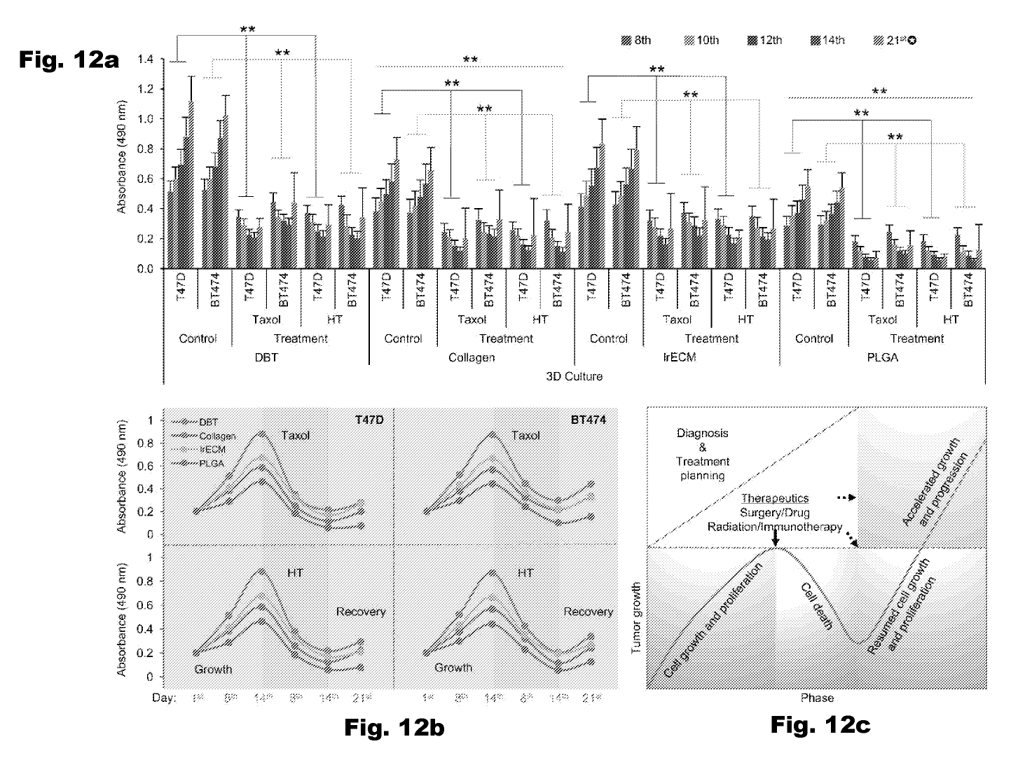

As the cancer cell-laden TMS in culture formed tumoroid (FIG. 3a, bottom panels) that resembled the solid tumor in vivo (FIGS. 7a, 7c), the performance of the TMS in testing anticancer drugs and in predicting treatment efficacies was investigated. Two ER-positive breast cancer cell lines T47D and BT474 (luminal subtype), which represent the most common type of breast cancer diagnosed to date, and respond to hormone therapy, were chosen for the experiments. First, the proliferation of the two types of cells on the selected kinds of scaffolds was assessed. The results showed that both the T47D and the BT474 cells proliferated faster on the DBT-TMS than those on the IrECM, collagen, and PLGA scaffolds (FIG. 11a), displaying a similar proliferation pattern to the MM231 cells grown on the scaffolds (FIGS. 3k-3n) but at a milder rate, which could be due to the slower proliferation nature of the T47D and the BT474 cells compared to the MM231 cells in vitro (not shown). Two anticancer drugs, Tamoxifen, a common ER antagonist drug in the active metabolite form of 4-hydroxytamoxifen (HT), and paclitaxel (Taxol), a cell mitotic arrest inducer drug, were selected for the tests. After growing the T47D or the BT474 cells in both 2D and 3D cultures for 7 days, the drugs were administered on alternative days starting on day 7 through day 13 (a total treatment of 4 times). As described above, the T47D and the BT474 cells proliferated quite well on the 3D scaffolds with distinguishable trends of growth between the scaffold groups (FIG. 12a), and more robust in the 2D cultures (FIG. 11b). Administration of the drugs time-dependently inhibited cell proliferation in both the 3D and the 2D cultures (FIG. 12a and FIG. 11b). However, the effect of the drug inhibition in the 3D cultures appeared to be less dramatic than that in the 2D cultures, which is consistent with previous reports (4, 5), with a better post-treatment recovery of cell proliferation after the 14th day as measured on the day 21st (FIG. 12a, FIG. 11b).

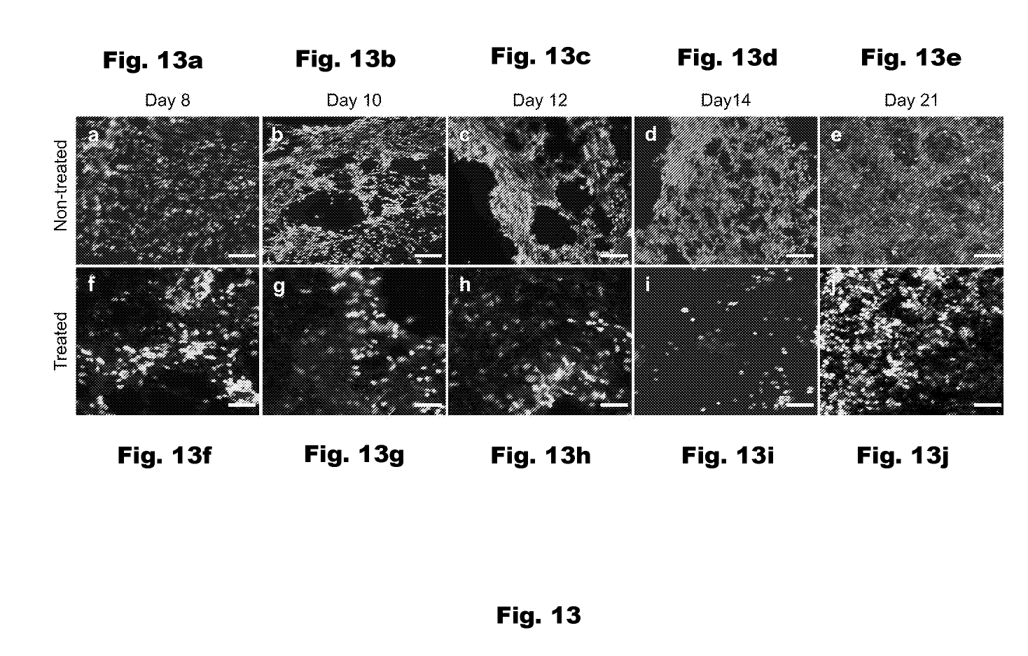

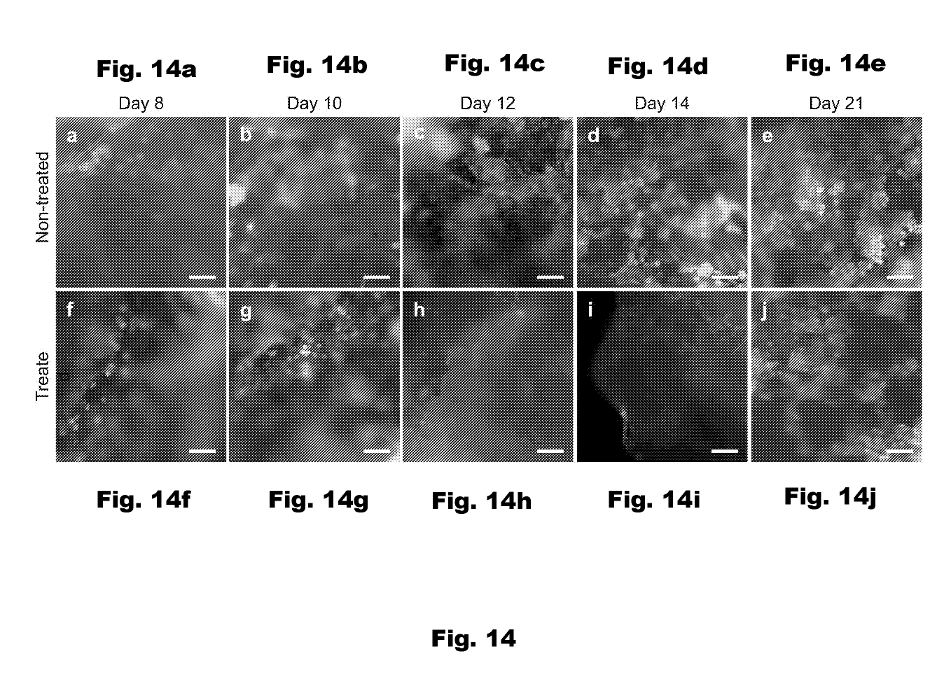

In agreement with a stronger support for cell survival and growth from the DBT-TMS than the other 3D scaffolds (FIGS. 3k-3n), cell proliferation on the DBT-TMS was the least inhibited by the drugs compared to those on the collagen, IrECM and PLGA scaffolds as depicted and compared on the proliferation/inhibition curve plots (FIG. 12b) summarized from FIG. 12a. Consistently, the post-treatment recovery of the cell proliferation on the DBT-TMS was better than those on the other scaffolds (FIG. 12b). Overall, the cells grown on the scaffolds generated using tissue-derived materials (DBT, collagen, and IrECM) showed faster post-treatment recovery than those cultured on the PLGA scaffolds (FIG. 12b). This trend also nicely represents clinical and animal model observations of tumor growth status during the disease development and intervention phases (56-58), where therapeutic applications cause cancer cell death and tumor shrinkage, and the tumor resumes growth and progresses (relapses) while the treatment is stopped or discontinued (FIG. 12c). When Live/Dead staining was performed on the cross sections of the scaffold cultures collected at different time points of the experiments, as illustrated by the results from the T47D-laden DBT-TMSs (FIGS. 13a-13j) and the PLGA scaffolds (FIGS. 14a-14j), the robust cell proliferation and clustering were markedly attenuated over the period of HT or Taxol treatment. A recovery of the proliferative phenotype was detected on Day 21, similar to what was seen in the proliferation experiments (FIGS. 13, 14 and FIGS. 12a, 12b). These data collectively indicate that TMS is a competent tissue-mimicry culture system suitable for anticancer drug screening. In conclusion, TMS represents an advanced native ECM-based 3D culture model that can be tailored for systemic biomedical research ranging from molecular mechanistic studies to animal models.

A tissue culture system that closely mimics the mechanical and pathophysiological properties of living tissues, is laboratory or research bench-fabricable, and can be tailored according to the experimental needs will greatly facilitate biomedical research including cancer studies. The TMS system that has been systemically validated satisfies the current needs for such a model. The structural and compositional mimicry of tissue ECM has marked the TMS an ideal system for solid tumor modeling, but also for modeling many types of tissues and organs and regeneration of skin and breast tissue, especially under situations where scaffolds generated from host or donor cells for autograft or allograft implantation are desired. For instance, culturing stem cells on the TMS and inducing tissue-specific cell expansion will be very useful for regenerative medicine. The TMS-supported cancer cell growth into tumoroid in 3D culture in vitro clearly mimicked the tumor formation in vivo in terms of histological characteristics and cell distribution within the ECM (FIGS. 3g-3j vs. FIG. 7c i-iv and FIGS. 8a-8d). Additionally, by introducing a naturally existing native cross-linker, tyrosinase, into the TMS hydrogel, a stiffness-controllable cross-linking of ECM proteins was achieved that facilitated the fabrication of a multilayered compartmental co-culture system containing both solid porous and hydrogel forms of ECM using the same tissue material. As the proper ECM stiffness or compliance is essential to signaling regulation of cancer cell attachment and migration (26, 27), the concentration-adjustable addition of tyrosinase provides flexibility for the generation of TMS with desired stiffness. Co-culture with compartmentalized TMS allows dynamic observation of the proliferation, interaction, migration and invasion of different cell populations within a single system that mimics the tissue microenvironment (FIG. 5). Importantly, molecular mechanisms involved in the regulation of these processes in the tissue microenvironments can be dissected using the multilayered 3D co-culture system coupled with the techniques of molecular biology and biochemistry that is otherwise impossible to achieve using the current 2D cultures and animal models.

The current embodiments demonstrate that the TMS has excellent biocompatibility with the host tissue that supports cancer cells to expand and integrate into surrounding tissues (FIG. 7c i-iv and FIGS. 8a-8d), where they can potentially stimulate fibroblasts to proliferate, differentiate into cancer associated fibroblasts (56) (FIG. 7c v-viii and FIGS. 8e-8h), and generate collagen for the migration and spread of cancer cells (46). The striking connective tissue-like ECM and clusters of microvessels within the TMS-supported tumors clearly distinguished the formed tumor tissue from normal breast tissue structures (FIG. 7c i-iv and FIGS. 8a-8d), where rich adipose and less connective tissues are present (47). These tumor tissue characteristics were well reflected by the phenotypes seen in cancer cells growing on the porous TMS alone (FIGS. 3g-3j) and their co-culture with fibroblasts (FIGS. 5f-5i). On the other hand, the host fibroblasts, endothelial cell and other cells are able to infiltrate into the TMS, entwine with the cancer cells (FIG. 7c v-viii and FIGS. 8e-8h), enhance angiogenesis (49, 62), and promote cancer cell proliferation through secretion of growth and other tumor promoting factors (46, 50). In addition, the biodegradability of the native tissue- and cell ECM-derived TMSs is better than that of the PLGA scaffold (FIG. 7a, upper panels). These properties of the TMS apparently contribute to the replacement of the degrading exogenous ECM by the host ECM that is built up by fibroblasts, macrophages, endothelial cells and other cells in the vicinity of the cancer locus besides the cancer cells. The new cancer cell-oriented stroma is able to induce desmoplastic differentiation and morphological changes of normal fibroblasts (29), stimulate tumorigenesis of the initiated epithelial cells (30), and is dynamically modified with alterations in inter- and intracellular signaling that further provide an improved tumor microenvironment (51) essential for expression of tumor promoting genes and accelerated cell migration, invasion (52, 53), and tumor growth (54). The excellent biocompatibility and biodegradability of the tissue ECM-based scaffolds (from both the mammary tissue and MM231 cells in this study) substantiates the initiation of solid tumors from cancer cells and quick expansion into a tumor mass. The necessity of a favorable stroma niche for the establishment of a tumor is also supported by the observation that the colonization of the metastatic cancer stem cells in a secondary site requires secretion of select signaling enzyme(s) into the ECM by local fibroblasts (55).

The looser intra-tumoral structures within the MM231-TMS-supported tumors could be a reflection of the under-developed tumor ECM (FIG. 7c ii, iv). The fibroblasts and endothelial cells recruited into the MM231-TMS need to be more active and proliferative to meet the pace for stroma and microvessel generation. This could be the reason that these cells were co-stained by Ki-67 and HER2 (FIG. 7c vi, viii and FIGS. 8f, 8h). Intriguingly, coating the cancer cell-laden TMSs with a layer of hydrogel decreased the sized of the tumors (FIG. 7a, bottom panels). This phenotype may in part be due to the physical constraint of the gel on the growth of the tumors and to the blockage of nutrient and O₂ supply, which were supported by the outward growth of the cancer cell population into the gel layer of the TMS (FIG. 7c vii, viii and FIGS. 8g, 8h). However, it cannot fully explain the deficient development of tumors out of the PLGA-scaffold. Since the normal breast-associated fibroblasts inhibit proliferation of the transformed epithelial cells (33), it could well be that the normal tissue-generated ECM also impedes the tumor development.

Another remarkable feature on the cross sections of the TMS-supported tumors is the microvessels distributed close to the outer sections of the tumors (FIG. 7c and FIGS. 8a-8h). Clearly, the degree of the tumor vascularization is proportional to the tumor size and the tumor cells around the vascularized regions. It seems that a better tumor ECM architecture is linked to enhanced-microvessel formation although the functional capability of these microvessels needs to be further investigated. These data are consistent with the observation that the cancerous breast fibroblasts can induce angiogenesis (48). These results collectively reiterate the critical role of the tissue microenvironment and ECM in tumor formation and progression.

This tumor biomarker staining on the TMS cross sections from both the 3D culture and the animal samples (FIG. 5c and FIG. 7c v-viii) matches very well with the clinical data (63). The pioneering staining results not only indicate that the TMS system supports the expression of the common tumor biomarkers, but also serve as a foundation for future exploration of novel tumor markers and for screening of the biomarkers of interest. Additionally, the system can be used in molecular mechanistic studies, where a specific regulation of tumor development is involved, and in large scale proteomics or genomics profiling for global changes of the molecules within the tumors. Further, the native tissue ECM-mimicry and potent cell growth-supporting properties (FIGS. 1d-1e and FIGS. 3c-3n), the capability of co-culturing multiplex cell types in separate yet free-accessing compartments and the animal tissue implantable feature make the TMS system a platform for therapeutic drug testing or screening at desired levels depending on the experimental needs. The efficacies of two common anticancer drugs, HT and Taxol, in the cell-laden TMS cultures were tested and a lower response of the cancer cells to the drugs was observed when compared to the other 3D culture conditions possibly due to the better cell survival and growth on the TMS (FIG. 12c and FIGS. 3k-3n). The resistance of the cancer cells grown on the TMS to the drugs also endow the cells a stronger post-treatment recovery (FIG. 12c), reminiscent of the clinical relapse of the cancers. The TMS is therefore also useful in predicting the prognosis of disease and in providing suggestions for additional therapeutics after the initial application of therapeutics. Taken together, the TMS represents an advance generation of 3D culture model that closely mimics the native tissue microenvironment, and allows systemic study of human tumors. These advantages of the TMS is encouraging in keeping the consistency of the research observations and in producing more clinically relevant data for a better translational outcome.

### Example 6: Protein Content of Porcine-Derived ECM

The protein content of porcine-derived extracellular matrix extract (from mammary and skin tissue) was analyzed by mass spectrometry using the methods described in Example 1. Table 3 shows the spectrum counts of the most abundant proteins detected in porcine mammary tissue extracellular matrix extract.

TABLE 3
Pig mammary tissue ECM major proteins

| | | | Spectrum |
|---|---|---|---|
| | # | Protein | Count |
| Collagen | 1 | Collagen type I alpha 2 chain | 546 |
| | 2 | Collagen type III alpha 1 chain | 369 |
| | 3 | Collagen type I alpha--1 chain | 176 |
| | 4 | Collagen type VI alpha 3 chain | 121 |
| | 5 | Collagen type II alpha 1 chain | 46 |
| | 6 | Collagen type XIV alpha 1 chain | 40 |
| | 7 | Collagen type VI alpha 2 chain | 34 |
| | 8 | Collagen type V alpha 2 chain | 25 |
| | 9 | Collagen type V alpha 1 chain | 23 |
| | 10 | Collagen type VI alpha 1 chain | 15 |
| | 11 | Collagen type XII alpha 1 chain | 8 |
| | 12 | Collagen type IV alpha 2 chain | 3 |
| | 13 | Collagen type VI alpha 5 chain | 3 |
| | 14 | Collagen type IV alpha 1 chain | 1 |
| | 15 | Collagen type V alpha 3 chain | 1 |
| Glycoprotein & | 16 | Fibrillin 1 | 33 |
| Proteoglycan/GAG | 17 | Lumican | 12 |
| | 18 | Tenascin--X | 6 |
| | 19 | Decorin | 5 |
| | 20 | Mimecan--like | 5 |
| | 21 | Asporin | 5 |
| | 22 | Olfactomedin--like protein 1--like | 5 |
| | 23 | Perlican | 5 |
| | 24 | Laminin subunit gamma 1 | 4 |
| | 25 | Versican | 3 |
| | 26 | Fibrinogen alpha chain | 3 |
| | 27 | Biglycan | 3 |
| | 28 | Fibrinogen beta chain | 1 |
| | 29 | Fibrinogen gamma chain | 1 |
| | 30 | Laminin subunit beta 1 | 1 |
| | 31 | Prolargin--like | 1 |
| | 32 | EMILIN 1 | 1 |
| | 33 | Fibulin--like extracellular matrix | 1 |
| | | protein 1 | |
| Myosin | 34 | Myosin--1 | 110 |
| | 35 | Myosin--4 | 101 |
| | 36 | Myosin--2 | 97 |
| | 37 | Myosin--7 | 20 |
| | 38 | Myosin light chain 2 | 14 |
| | 39 | Myosin--9 | 13 |
| | 40 | Tropomyosin alpha 1 chain | 9 |
| | 41 | Myosin light chain 1 | 7 |
| | 42 | Myomesin 2 | 5 |
| | 43 | Moesin | 3 |
| | 44 | Myosin--Ic | 2 |
| Other ECM | 45 | Titin | 9 |
| Proteins | 46 | Dermatopontin | 3 |

Table 4 shows the spectrum counts of the most abundant proteins detected in porcine skin tissue extracellular matrix extract.

TABLE 4
Pig skin tissue major ECM proteins

| | | | Spectrum |
|---|---|---|---|
| | # | Protein | Count |
| Collagen | 1 | Collagen type I alpha 1 chain | 1135 |
| | 2 | Collagen type I alpha 2 chain | 796 |
| | 3 | Collagen type V alpha 2 chain | 86 |
| | 4 | Collagen type II alpha 1 chain | 77 |
| | 5 | Collagen type V alpha 1 chain | 62 |
| | 6 | Collagen type VI alpha 3 chain | 43 |
| | 7 | Collagen type IV alpha 2 chain | 31 |
| | 8 | Collagen type XII alpha 1 chain | 18 |
| | 9 | Collagen type VI alpha 2 chain | 11 |
| | 10 | Collagen type VI alpha 1 chain | 18 |
| | 11 | Collagen type V alpha 3 chain | 11 |
| | 12 | Collagen type XIV alpha 1 chain | 10 |
| | 13 | Collagen type IV alpha 1 chain | 8 |
| | 14 | Collagen type VIII alpha 1 chain | 4 |
| | 15 | Collagen type XVI alpha 1 chain | 3 |
| | 16 | Fibrillin 1 | 100 |
| Glycoprotein & | 17 | Perlecan | 15 |
| Proteoglycan/GAG | 18 | Laminin subunit gamma 1 | 3 |
| | 19 | Lumican | 2 |
| | 20 | Tenascin--X | 2 |
| | 21 | Mimecan--like | 2 |
| | 22 | Fibrinogen alpha chain | 2 |
| | 23 | EMILIN 1 | 2 |
| | 24 | Asporin | 1 |
| | 25 | Versican | 1 |
| | 26 | Fibrinogen beta chain | 1 |
| | 27 | Laminin subunit beta 1 | 1 |
| | 28 | Fibulin--like extracellular matrix | 1 |
| | | protein 1 | |
| Myosin | 29 | Myosin--1 | 25 |
| | 30 | Myosin--4 | 23 |
| | 31 | Myosin--2 | 23 |
| | 32 | Myosin--9 | 9 |
| | 33 | Tropomyosin alpha 1 chain | 3 |
| | 34 | Myosin--Ic | 2 |
| Other ECM | 35 | Dermatopontin | 6 |
| Proteins | 36 | Titin | 1 |

As will be understood by one of ordinary skill in the art, each embodiment disclosed herein can comprise, consist essentially of, or consist of its particular stated element, step, ingredient or component. Thus, the terms “include” or “including” should be interpreted to recite: “comprise, consist of, or consist essentially of.” As used herein, the transition term “comprise” or “comprises” means includes, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment. As used herein, a material effect would cause a reduction in the ability of a 3D culture of cells to proliferate as disclosed herein.

It is understood that for any given component or embodiment described herein, any of the possible candidates or alternatives listed for that component may generally be used individually or in combination with one another, unless implicitly or explicitly understood or stated otherwise. It is to be noted that as used herein, the term “adjacent” does not require immediate adjacency. Moreover, it is to be appreciated that the figures, as shown herein, are not necessarily drawn to scale, wherein some of the elements may be drawn merely for clarity of the invention. Also, reference numerals may be repeated among the various figures to show corresponding or analogous elements. Additionally, it will be understood that any list of such candidates or alternatives is merely illustrative, not limiting, unless implicitly or explicitly understood or stated otherwise.

Unless otherwise indicated, all numbers used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

The particulars shown herein are by way of example and for purposes of illustrative discussion of the preferred embodiments of the present invention only and are presented in the cause of providing what is believed to be the most useful and readily understood description of the principles and conceptual aspects of particular embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Definitions and explanations used in the present disclosure are meant and intended to be controlling in any future construction unless clearly and unambiguously modified in the examples or when application of the meaning renders any construction meaningless or essentially meaningless. In cases where the construction of the term would render it meaningless or essentially meaningless, the definition should be taken from Webster's Dictionary, 3rd Edition or a dictionary known to those of ordinary skill in the art, such as the Oxford Dictionary of Biochemistry and Molecular Biology (Ed. Anthony Smith, Oxford University Press, Oxford, 2004).

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described. All publications, patents and patent applications cited in this specification are incorporated herein by reference in their entireties as if each individual publication, patent or patent application were specifically and individually indicated to be incorporated by reference. While the foregoing has been described in terms of various embodiments, the skilled artisan will appreciate that various modifications, substitutions, omissions, and changes may be made without departing from the spirit thereof.

### REFERENCES

1. G. Rijal, W. Li, Biomaterials 81, 135-156 (2016).
2. J. Debnath, J. S. Brugge, Nature reviews. Cancer 5, 675-688 (2005).
3. T. Vargo-Gogola, J. M. Rosen, Nature reviews. Cancer 7, 659-672 (2007).
4. M. Hakanson, M. Textor, M. Charnley, Integr Biol (Camb) 3, 31-38 (2011).
5. F. Deiss, et al. Anal Chem 85, 8085-8094 (2013).
6. P. A. Kenny, et al. Mol Oncol 1, 84-96 (2007).
7. W. Li, et al., Circ Res 100, 79-87 (2007).
8. M. Zanoni, et al., Sci Rep 6, 19103 (2016).
9. K. Y. Lee, D. J. Mooney, Chem Rev 101, 1869-1879 (2001).
10. T. P. Kraehenbuehl, R. Langer, L. S. Ferreira, Nat Methods 8, 731-736 (2011).
11. M. C. Kibbey, Journal of tissue culture methods, 227-230 (1994).
12. R. W. Orkin, et al. The Journal of experimental medicine 145, 204-220 (1977).
13. S. Vukicevic, H. K. Kleinman, F. P. Luyten, A. B. Roberts, N. S. Roche, A. H. Reddi, Experimental cell research 202, 1-8 (1992).
14. H. K. Kleinman, et al. Biochemistry 25, 312-318 (1986).
15. O. W. Petersen, et al. PNAS 89, 9064-9068 (1992).
16. S. F. Badylak, Transpl Immunol 12, 367-377 (2004).
17. F. Pati, et al. Nature communications 5, 3935 (2014).
18. J. A. DeQuach, et al. PLoS One 5, e13039 (2010).
19. D. J. Wainwright, Burns 21, 243-248 (1995).
20. Gerecht-Nir S, et al. Biotechnol Bioeng 88, 313-320 (2004).
21. Gerecht S, B et al. PNAS 104, 11298-11303 (2007).
22. Li Z, et al. Biomaterials 31, 404-412 (2010).
23. Pati F, et al. Nature communications 5, 3935 (2014).
24. P. M. Crapo, T. W. Gilbert, S. F. Badylak, Biomaterials 32, 3233-3243 (2011).
25. J. Radosavljevic, et al. Mol Nutr Food Res 58, 635-646 (2014).
26. J. C. Friedland, M. H. Lee, D. Boettiger, Science 323, 642-644 (2009).
27. M. J. Paszek, V. M. Weaver, J Mammary Gland Biol Neoplasia 9, 325-342 (2004).
28. W. D. Lu, L. Zhang, et al. PLoS One 9, e103672 (2014).
29. M. D. Amatangelo, et al. Am J Pathol 167, 475-488 (2005).
30. A. F. Olumi, et al. Cancer research 59, 5002-5011 (1999).
31. J. T. Camp, F. Elloumi, E. Roman-Perez, J. Rein, D. A. Stewart, J. C. Harrell, C. M. Perou, M. A. Troester, Mol Cancer Res 9, 3-13 (2011).
32. J. J. Campbell, et al. PLoS One 6, e25661 (2011).
33. A. Sadlonova, Z. et al. Frost, Breast cancer research: BCR 7, R46-59 (2005).
34. P. Correa de Sampaio, et al. PLoS One 7, e30753 (2012).
35. H. Jaganathan, J. Gage, F. Leonard, S. Srinivasan, G. R. Souza, B. Dave, B. Godin, Sci Rep 4, 6468 (2014).
36. K. E. Sung, et al. PLoS One 8, e76373 (2013).
37. H. Hwang, et al. Biomed Microdevices 15, 627-634 (2013).
38. S. Ladet, L. David, A. Domard, Nature 452, 76-79 (2008).
39. X. Fang, S. et al. PLoS One 8, e75187 (2013).
40. M. E. Todhunter, et al. Nat Methods 12, 975-981 (2015).
41. A. Urruticoechea, I. E. Smith, M. Dowsett, J Clin Oncol 23, 7212-7220 (2005).
42. B. Kocaturk, H. H. Versteeg, J Vis Exp, (2015).
43. E. Iorns, et al., PLoS One 7, e47995 (2012).
44. C. Fischbach, et al. Nat Methods 4, 855-860 (2007).
45. D. Ohlund, E. Elyada, D. Tuveson, The Journal of experimental medicine 211, 1503-1523 (2014).
46. P. Cirri, P. Chiarugi, Am J Cancer Res 1, 482-497 (2011).
47. N. A. Bhowmick, et al. Nature 432, 332-337 (2004).
48. H. M. Jensen, et al. Science 218, 293-295 (1982).
49. E. Y. Lin, et al. Cancer research 66, 11238-11246 (2006).
50. G. Su, et al. PLoS One 7, e46685 (2012).
51. C. J. Olsen, et al. BMC cancer 10, 444 (2010).
52. S. A. Newman, et al. Science 228, 885-889 (1985).
53. T. W. Ridky, et al. Nat Med 16, 1450-1455 (2010).
54. P. A. Kenny, M. J. Bissell, Int J Cancer 107, 688-695 (2003).
55. I. Malanchi, A. et al. Nature 481, 85-89 (2012).
56. M. V. Patrone, et al. Oncology (Williston Park) 25, 38-43, 46 (2011).
57. C. Ferte, et al. Eur Urol 65, 713-720 (2014).
58. N. V. Jordan, et al. Nature 537. 102-106 (2016).
59. H. C. Ott, et al. Nat Med 14, 213-221 (2008).
60. G. M. Williams, T. J. Klein, R. L. Sah, Acta biomaterialia 1, 625-633 (2005).
61. M. D. Buschmann, et al. J Cell Sci 108 (Pt 4), 1497-1508 (1995).
62. Izumi Y, Xu L, di Tomaso E, Fukumura D, Jain R K. Nature 416, 279-280 (2002).
63. Urruticoechea A, Smith I E, Dowsett M. J Clin Oncol 23, 7212-7220 (2005).

1-68. (canceled)

69. A composition comprising tissue-derived extracellular matrix (ECM) extract, wherein the extract comprises a protein content of less than about 45% laminin, and wherein the extract is decellularized and delipidated.

70. The composition of claim 69, wherein the extract is in a powder form.

71. The composition of claim 69, wherein the less than about 45% laminin is less than 40%, less than 30%, less than 20%, less than 10%, or less than 5% laminin.

72. The composition of claim 69, wherein the extract does not include synthetic polymers.

73. The composition of claim 69, wherein the extract is derived from a mammal.

74. A hydrogel tissue matrix scaffold (TMS) comprising a hydrogel of a composition of claim 69.

75. A porous tissue matrix scaffold (TMS) comprising a decellularized, delipidated and dehydrated tissue-derived extracellular matrix (ECM) extract.

76. The TMS of claim 75, wherein the TMS comprises a protein content of less than about 45% laminin.

77. The TMS of claim 76, wherein the TMS does not include synthetic polymers.

78. A multilayered TMS comprising a hydrogel TMS of claim 74.

79. A multilayered TMS comprising a porous TMS of claim 75.

80. A method of producing the hydrogel TMS of claim 74 comprising:
obtaining a sample derived from a fresh, homogenized tissue;
decellularizing and delipidating the tissue;
lyophilizing the decellularized and delipidated tissue to obtain a powder;
suspending the powder in a buffer to form the hydrogel; and
allowing the hydrogel to solidify;
thereby producing the hydrogel TMS.

81. A method of producing the porous TMS of claim 75 comprising:
obtaining a sample derived from a fresh, homogenized tissue;
decellularizing and delipidating the tissue;
lyophilizing the decellularized and delipidated tissue to obtain a powder;
suspending the powder in a buffer to form a liquid;
extracting the protein from the liquid to form a liquid ECM extract; and
concentrating the liquid ECM extract to form a hydrogel;
dehydrating the hydrogel to form a porous solid;
thereby producing a porous TMS.

82. A method of preparing a three-dimensional cell culture, the method comprising seeding cells or tissue on or into the hydrogel TMS of claim 74 and allowing the cells to proliferate.

83. A method of preparing a three-dimensional cell culture, the method comprising seeding cells or tissue on or into the porous TMS of claim 75 and allowing the cells to proliferate.

84. A method of generating tissue comprising
seeding a multilayer TMS with cells;
adding growth medium to the seeded multilayer TMS; and
allowing the cells to proliferate on the multilayer TMS, thereby generating tissue.

85. A method of preparing a tumoroid for in vivo implant, the method comprising
seeding the hydrogel TMS of claim 74 with cancer cells,
preculturing the cancer cells in the hydrogel TMS in vitro to allow a tumoroid to form.

86. A method of preparing a tumoroid for in vivo implant, the method comprising
seeding the porous TMS of claim 75 with cancer cells,
preculturing the cancer cells in the porous TMS in vitro to allow a tumoroid to form.

87. A kit for producing a TMS comprising the composition of claim 69, and reagents and/or instructions for producing a TMS from the composition.

88. A kit for generating tissue, the kit comprising:
a porous TMS;
(i) the composition of claim 69 and instructions for producing a hydrogel TMS from the composition of claim 69, or (ii) a hydrogel TMS; and
instructions for seeding cells in or on the porous TMS and the hydrogel TMS.
